(19) United States
(12) Patent Application Publication
(10) Pub. No.: US 2021/0046101 A1
(43) Pub. Date: Feb. 18, 2021

(54) COMBINATION THERAPEUTICS

(71) Applicant: UCL BUSINESS LTD, London (GB)

(72) Inventors: Henning WALCZAK, London (GB); Lucia TARABORRELLI, London (GB); Nieves PELTZER, London (GB)

(21) Appl. No.: 16/964,062

(22) PCT Filed: Jan. 22, 2019
(86) PCT No.: PCT/EP2019/051442

§ 371 Date: Jul. 22, 2020

(30) Foreign Application Priority Data
Jan. 22, 2018 (GB) 1800994.4

(51) Int. Cl. A61K 31/7088; A61P 29/00; C07K 16/28; A61K 31/56; A61K 38/17; A61K 35/17

(52) U.S. Cl. CPC A61K 31/7088; A61P 29/00; C07K 16/2818; A61K 45/06; A61K 31/56; A61K 38/177; A61K 35/17; C07K 16/2827

(57) ABSTRACT

The invention provides novel treatments (methods, uses and compositions) for treating inflammatory disease based on administering to the subject a combination of at least three agents targeting multiple death-receptor inducing systems, the combination comprising: (1) a first agent that neutralises the receptor TNFR1 or a ligand thereof; and (2) a second agent that neutralises either of: (2a) TRAIL-R, or a ligand thereof; or (2b) CD95, or a ligand thereof; and: (3) a third agent that neutralises any of: (3a) TLR3, or TLR4, or a ligand of either; or (3b) a further, different, receptor which is a TNF Receptor superfamily member shown in Table 1, or a ligand thereof; (3c) Caspase; (3d) RIPK1.

### TECHNICAL FIELD

The present invention relates generally to improved methods and materials for use in treating diseases with TNF inhibitors or related agents.

### BACKGROUND ART

Tumour necrosis factor (TNF) is a major inducer of inflammation¹ and patients suffering from many different auto-immune diseases can be treated successfully with TNF inhibitors, either alone or in combination with other drugs².

However, therapy with TNF inhibitors is not always effective; e.g., only about 50% of patients suffering from rheumatoid arthritis (RA), about 65% of patients with psoriasis and about 60-80% of patients with inflammatory bowel disease (IBD) respond to treatment with TNF inhibitors^{3,4}.

Furthermore, there are many other diseases where patients do not benefit from treatment with TNF inhibitors⁵.

Croft and Siegel (Nature Reviews Rheumatology 13.4 (2017): 217-233) discuss the potential of certain members of the TNF superfamily (TNFSF) as targets for future therapy of rheumatic diseases. They note TNFSF members initiate several processes, including immune activation, tissue inflammatory responses and cell death or suppression. In relation to blocking tissue inflammation, for example in patients with RA who were unresponsive to TNF blockers, there is a particular emphasis on neutralising TWEAK and LIGHT members of the TNFSF, in addition to TNF (page 229).

JP2002114800 relates to peptides based on receptor sequences and which are reported to have inhibitory activity against TNF, TRAIL and FasL. These are said to be useful for inhibiting apoptosis and inflammation caused by these ligands.

Nevertheless, new therapeutic strategies are required for patients who suffer from diseases including, but not limited to (auto-)inflammatory, auto-immune and other diseases, such as the ones listed above, driven by mechanisms beyond TNF. The provision of such novel treatments would provide a contribution to the art.

### DISCLOSURE OF THE INVENTION

The present inventors have used novel models of inflammatory disease to provide novel combinatorial therapies for such diseases based on the inhibition of cell death mediated by combinations of agents which blockade (or otherwise inhibit) ligands or their receptors, optionally in conjunction with blockading (or otherwise inhibiting) mediators of extrinsic apoptosis and necroptosis.

Examples of such ligands include members of the TNF superfamily.

In addition to TNF itself, other TNF superfamily members including, but not limited to, lymphotoxin (LT)-α, LT-β, CD95 ligand (CD95L; also known as FasL or APO-1L), TRAIL (also known as Apo2L), TWEAK and TL1A, as well as ligands for pattern recognition receptors (PPRs) including, but not limited to, the PRR known as toll-like receptor (TLR) 3, are able to induce cell death^{10,11}.

In particular the inventors have shown that combination interventions in relation to such targets can lead to synergistic effects.

By way of non-limiting example, combined ablation or impairment of TNF superfamily receptors (TNFR1, TRAIL-R and CD95) in a mouse inflammatory model completely prevented inflammation, whereas targeting these receptors individually did not have that effect.

By further way of non-limiting example, combining ablation of TNF superfamily receptors TNFR1 and TRAIL-R with TLR3 (a toll-like receptor) in the mouse inflammatory model provided improved amelioration of inflammation, compared to targeting only two of these receptors individually (TNFR1 with TRAIL-R, or TNFR1 with TLR3).

Other findings of the inventors in support of the present invention are described hereinafter.

TABLE 1
TNF Receptor superfamily members
and corresponding cognate ligands

| Member | Synonyms | Gene | Ligand(s) |
|---|---|---|---|
| Tumor necrosis | TNFR1, CD120a | TNFRSF1A | TNF (also known |
| factor | | | as TNF-alpha) |
| (TNF) receptor | | | Lymphotoxin |
| (TNFR) 1 | | | (LT)- alpha (also |
| | | | known as TNF- |
| | | | beta) |
| Lymphotoxin beta | LTBR, CD18 | LTBR | LT-alpha, LT- |
| receptor | | | beta |
| | | | LIGHT |
| CD95 | APO-1, Fas | APT1 | CD95L (also |
| | | | known as FasL |
| | | | or APO-1 L) |
| TRAIL-R1 | Death receptor 4, | TNFRSF10A | TRAIL (also |
| | Apo-2, CD261 | | known as Apo2L) |
| TRAIL-R2 | , Death receptor 5, | TNFRSF10B | |
| | CD262 | | |
| TRAIL-R3 | , Decoy receptor 1, | TNFRSF10C | |
| | LIT, TRID, CD263 | | |
| TRAIL-R4 | Decoy receptor 2, | TNFRSF10D | |
| | TRUNDD, CD264 | | |
| Death receptor 6 | CD358 | TNFRSF21 | |
| Death receptor 3 | Apo-3, TRAMP, | TNFRSF25 | TL1A |
| | LARD, WS-1 | | |
| FN14 | TWEAK receptor, | TNFRSF12A | TWEAK |
| | CD266 | | |

These findings of the inventors demonstrate that multiple death-receptor inducing systems (TNFR1, TRAIL-R and\or CD95, plus a third target) can act in combination to contribute to inflammation-associated diseases, that they can indeed compensate for each other and that, thus, treatment of these diseases may be improved by blocking such systems in combination.

As explained below, inhibition of cell death mediated by these receptors may be advantageously combined with inhibition of the activity of Caspases, preferably with inhibition of receptor interacting protein kinase 3 (RIPK3) and/or MLKL.

These combination therapies are explained in more detail hereinafter.

This has particular implications for diseases in which inhibitors of e.g. TNF (or other TNF superfamily ligands such as LT-8) have not worked as single agents. Examples of diseases include inflammation and inflammation-associated diseases including, but not limited to, auto-immune diseases, neuro-inflammatory diseases, neuro-degenerative diseases, ischaemic diseases, sepsis, and cancer.

Aspects of the invention provide combinations of agents that neutralise or decrease the biological activity of TNF Receptor superfamily members or their respective ligands, along with other receptors or ligands able to induce cell death such as TLR3 or TLR4 and its ligands and\or mediators of extrinsic apoptosis and necroptosis in methods, or in the manufacture of a medicaments, for the treatment of the diseases described herein. Such agents may decrease the biological activity of (for example) TNF/LT-α, TRAIL, CD95L, or TNFR1, TRAIL-Rs, CD95, RIPK1, TLR3, TLR4, Caspase-8, RIPK3 and MLKL.

The methods of the invention may neutralize death receptors or death ligands to inhibit, or result in inhibition of, cell death, with therapeutic benefit in diseases of inflammation. This is achieved by inhibition of, or prevention of activation of cell death by, TNF/LT-α, TRAIL, CD95L, dsRNA, LPS, and/or a TNFR1, TRAIL-R, CD95, TLR3, TLR4, or inhibition, or prevention of activation of cell death by, RIPK1, RIPK3, MLKL or caspase-8.

It has previously been shown that TNF can drive inflammation via inducing aberrant cell death^{6,7}. Prior to that the dogma had been that TNF drives inflammation and auto-immunity by inducing aberrantly high levels of gene activation. On the basis of that discovery, it has been proposed that in patients with a TNF-induced cell death aetiology of disease, TNF inhibition could work by inhibiting the aberrant TNF-induced cell death rather than the TNF-induced gene activation⁸.

Furthermore it has previously been shown that loss of Caspase-8 and RIPK3/MLKL prevent dermatitis in certain inflammatory models. Furthermore it has previously been suggested that lethal dermatitis in a mouse model of inflammation was provoked by excessive Caspase-8-driven apoptosis which was mediated by, but also independently of TNFR1, suggesting a pathology resulting from TNFR1-independent and also RIPK1 kinase- and Caspase-8 dependent apoptosis (see e.g. Abstract presented at 15th TNF international conference, May 20-23, 2015, Ghent, Belgium).

However, those earlier disclosures did not teach or suggest the combination therapies of the present invention.

Thus according to one aspect of the invention there is provided a method for treating inflammatory disease in a subject, the method comprising administering to the individual a combination treatment of at least 3 agents, the combination comprising:

(1) a first agent that neutralises the receptor TNFR1 or a ligand thereof; and

(2) a second agent that neutralises either of: (2a) TRAIL-R, or a ligand thereof; or (2b) CD95, or a ligand thereof; and:

(3) a third agent that neutralises any of: (3a) TLR3, or TLR4, or a ligand of either; or (3b) a further, different, receptor which is a TNF Receptor superfamily member shown in Table 1, or a ligand thereof; (3c) Caspase; or (3d) RIPK1.

“Neutralises” in this context will be understood to mean modulates a biological activity of, either directly (for example by binding to the relevant target) or indirectly. As used herein, the term “biological activity” means any observable effect resulting from the interaction between the protein\receptor (binding partners). Non-limiting examples of biological activity in the context of the present invention include signalling and regulation of the genes discussed herein e.g. those involved in cell apoptosis or necroptosis.

“Neutralises” does not imply complete inactivation. The modulation is generally inhibition i.e. a reduction or diminution in the relevant biological activity by comparison with the activity seen in the absence of the agent.

Neutralisation is typically achieved by (i) preventing or inhibiting the ligand from binding to the receptor; (ii) disrupting the receptor/ligand complex resulting from such binding.

The invention further provides a method of enhancing the therapeutic effectiveness of any of the agents (e.g. the first agent) for treating an inflammatory disease in a subject, the method comprising administering to the individual the other two agents.

In one embodiment the first agent neutralises TNF and/or LT-α. In one embodiment the first agent neutralises TNF.

In one embodiment the second agent neutralises any, or a combination, of the TRAIL-Rs, or neutralises TRAIL. In one embodiment the second agent neutralises TRAIL-R2.

In one embodiment the third agent neutralises CD95, or neutralises CD95L.

Thus the invention embraces the use of:

(1) an agent which neutralises TNF and/or LT-α;

(2) an agent which neutralises TRAIL-R or TRAIL;

(3) an agent which neutralises CD95L.

In another embodiment the third agent neutralises TLR3 or TLR4, or neutralises a ligand of TLR3 or TLR4.

In one embodiment the second agent neutralises CD95, or neutralises CD95L.

In one embodiment the third agent neutralises TLR3 or TLR4, or neutralises a ligand of TLR3 or TLR4.

Thus the invention embraces the use of:

(1) an agent which neutralises TNF and/or LT-α;

(2) an agent which neutralises CD95 or CD95L;

(3) an agent which neutralises TLR3.

In another embodiment the third agent neutralises one or more Caspases (e.g. Caspase 8 and/or Caspase 10), and a fourth agent is used which neutralises RIPK3 and\or MLKL.

In another embodiment the third agent neutralises LT-β.

In another embodiment the third agent neutralises RIPK1.

As explained below fourth and other additional agents may also be used.

For example, when not already included in the combination therapy, a fourth agent may neutralise one or more Caspases (e.g. Caspase 8), and an optional fifth agent may neutralise RIPK3 and/or MLKL.

Other additional agents include further anti-inflammatory biologic or anti-inflammatory chemical agents known in the art. In one embodiment the further anti-inflammatory biologic or chemical agent is an oral or topical corticosteroid.

Particular Example embodiments of the invention include:

Use of combinations of agents that neutralise one or more of TNF/LT-α, TRAIL, CD95L, dsRNA (binding to TLR3), LPS (binding to TLR4) and/or neutralise one or more of TNFR1/TRAIL-R/CD95/TLR3 and/or diminishes one or more interactions: TNF/LT-α/TNFR1, TRAIL/TRAIL-R, CD95L/CD95, dsRNA/TLR3, LPS/TLR4.

Use of agents which diminish the activity of RIPK1, RIPK3, MLKL or caspase-8 in combination with the above combinations.

Use of combinations of agents that prevent or inhibit the ligands TNF/LT-α, TRAIL, CD95L, dsRNA, LPS from binding to the receptors TNFR1, TRAIL-Rs, CD95, TLR3, TLR4, respectively, or disrupt a TNF/LT-α/TNFR1, TRAIL/TRAIL-R, CD95L/CD95, dsRNA/TLR3, LPS/TLR4 complexes resulting from such binding.

Use of agents that prevent or inhibit the activity of RIPK1, RIPK3, MLKL or caspase-8 resulting from the ligand-receptor binding described above.

### Examples of Agents

Examples of neutralising agents suitable for use in the invention are described in more detail hereinafter. They include small molecules, antibodies or fragments thereof that bind to and neutralise the receptor or ligand, single or double-stranded nucleotide (DNA, RNA (siRNA, miRNA, shRNA), PNA, DNA-RNA-hybrid molecule) that interfere with expression of the receptor or ligand.

Thus by way of non-limiting example the invention may use of combinations of agents that bind to TNFR1, TRAIL-Rs, preferably TRAIL-R1 and/or TRAIL-R2, CD95, TLR3 or TLR4—for example an antibody or fragment thereof that binds specifically to TNFR1, TRAIL-Rs, preferably TRAIL-R1 and/or TRAIL-R2, CD95, TLR3, TLR4, or a small molecule or fragment thereof that binds specifically to TLR3 or TLR4, neutralising their activity, for example which blocks receptor-mediated intracellular signalling.

The invention may use agents that bind to RIPK1, RIPK3, MLKL or caspase-8. For example a small molecule or fragment thereof that binds specifically to RIPK1, RIPK3, MLKL or caspase-8 neutralising their activity, for example which blocks kinase or protease activity.

The example may use agents each of which is a fusion protein comprising an extracellular or other domain of TNFR1, a TRAIL-R, preferably TRAIL-R2 or TRAIL-R1, CD95, TLR3, TLR4 or a portion thereof, fused to a portion of a human antibody, preferably an Fc domain, or a portion thereof, with or without the antibody hinge region, or a portion thereof.

The invention may use agents that are single- or double-stranded nucleotides (DNA, RNA (sIRNA, rhiRNA, shRNA), PNA, DNA-RNA-hybrid molecule) that interfere with TNF/LT-α, TRAIL, CD95L, and/or TNFR1, any of the TRAIL-Rs, preferably TRAIL-R1 and/or TRAIL-R2, CD95, TLR3, TLR4, and/or RIPK1, RIPK3, MLKL or caspase-8 expression, for example by binding to RNA transcripts such as to reduce expression.

Use of agents that decrease the biological activity of TNFR1, any of the TRAIL-Rs, preferably TRAIL-R1 and/or TRAIL-R2, CD95, TLR3, TLR4 by:

(a) decreasing the expression of the receptors;

(b) increasing receptors' desensitisation or receptors' breakdown;

(c) reducing interaction between TNF/LT-α, TRAIL, CD95L, dsRNA, LPS and the respective endogenous receptors;

(d) reducing receptors' mediated intracellular signalling;

(e) competing with endogenous receptor for TNF/LT-α, TRAIL, CD95L, dsRNA, LPS binding;

(f) binding to the receptor to block TNF/LT-α, TRAIL, CD95L, dsRNA, LPS binding; or

(g) binding to TNF/LT-α, TRAIL, CD95L, dsRNA, LPS preventing interaction with the receptors.

(h) reducing the kinase activity of RIPK1 and RIPK3;

(i) reducing the protease activity of caspase-8;

(j) reducing the expression of RIPK1, RIPK3, MLKL and/or caspase-8;

(k) reducing the interaction of RIPK1 with RIPK3 and/or caspase-8;

(l) reducing the interaction of RIPK3 with MLKL;

(m) reducing the intracellular signalling of RIPK1, RIPK3, MLKL and/or caspase-8

Inhibitors which act on the ligands recited in the claims are available commercially or are described herein.

Preferred inhibitors are shown in Table 2.

TABLE 2
Inhibitors which may be used in the invention

| Target | Inhibitor | References |
|---|---|---|
| TNF | Etanercept | (Croft and Siegel, 2017) |
| TNF | Infliximab | (Croft and Siegel, 2017), |
| | | U.S. Pat. No. 5,919,452 A |
| TNF | Adalimumab | (Croft and Siegel, 2017), |
| | | EP 0914157 B1 |
| TNF | Golimumab | (Croft and Siegel, 2017) |
| TNF | Certolizumab pegol | (Croft and Siegel, 2017), |
| | | WO 2013087912 A1 |
| TNF | TNF-kinoid | (Croft and Siegel, 2017), |
| | | WO 2007022813 A2 |
| CD95L | Asunercept (APG101) | (Wick et al., 2014), |
| | | EP 1447093 |
| | | A1, WO 2004071528 A1 |
| CD95L | FLINT | EP 1020521 A1 |
| CD95/ | antibody against CD95L or an | WO 2010006772 A3 |
| CD95L | antigen-binding fragment | |
| | thereof; soluble CD95 molecule | |
| TRAIL | TRAIL-R2-FC | WO2015001345 |
| TLR3 | TLR3 antagonist antibody | U.S. Pat. No. 8,153,583 B2 |
| TLR3 | Peptide-GNP hybrid | (Yang et al., 2016) |
| TLR3 | Small molecules | (Cheng et al., 2011) |
| TLR4 | TAK-242, Candesartan, | (Gao et al. 2017) |
| | Valsartan, Fluvastatin, | |
| | Simvastatin, Atorvastatin | |
| TLR4 | Antibodies - NI-0101 | (Gao et al. 2017) |
| TLR4 | Eritoran (E5564); miR-146a; | (Gao et al. 2017) |
| | miR-21; NAHNP; HDL-like NP; | |
| | Bare GNP; Glycolipid-coated | |
| | GNP; Peptide-GNP hybrid | |
| Caspases | emricasan | (Hoglen et al., 2004) |
| Caspases | GS-9450 | (Manns et al., 2010; Ratziu |
| | | et al., 2012) |
| LT-β | Baminercept | (St Clair et al., 2015) |
| MLKL | Ponatinib | (Fauster et al., 2015) |
| MLKL | pazopanib | (Fauster et al., 2015) |
| RIPK3 | Kongensin A | (Li et al., 2016) |
| RIPK3 | Celastrol | (Jia et al., 2015) |
| RIPK1 | GSK2982772 | (Harris et al., 2017) |

Some of these will be now described in more detail:

Blockade of TNF has been extensively used in the clinic and there are several inhibitors of TNF (signalling) available². Commercially available monoclonal TNF-neutralising antibodies or recombinant proteins are, for example: Etanercept/Enbrel (Amgen, Pfizer) which is a TNFR2-immunoglobulin fusion protein that neutralises TNF and LT-α; Infliximab/Remicade from (Johnson & Johnson); Adalimumab/Humira from (AbbVie Inc.); Golimumab/Simponi (Janssen Biotech); Certolizumab/Cimzia (UCB).

For the inhibition of LT-β, Baminercept which is LT-β receptor-immunoglobulin fusion protein is available.

The invention may utilise an agent which decreases the biological activity of any, or a combination, of the TRAIL-Rs, preferably TRAIL-R1 and/or TRAIL-R2, or TRAIL by:
(a) decreasing the expression of the receptor(s);
(b) increasing receptor desensitisation or receptor breakdown;
(c) reducing interaction between TRAIL and the receptor(s) which is (are) (an) endogenous receptor(s);
(d) reducing receptor-mediated intracellular signalling;
(e) competes with endogenous receptor(s) for TRAIL binding;
(f) binds to the receptor(s) to block TRAIL binding; or
(g) binds to TRAIL preventing interaction with the receptor(s).

For agents which bind to and neutralise TRAIL, an antibody or fragment thereof that binds to and neutralises TRAIL.

Commercially available monoclonal TRAIL-neutralizing antibodies are, for example anti-human TRAIL clone 2E5 from Enzo (http://www.enzolifesciences.com/ALX-804-296/trail-human-mab-2e5/) and Anti-TRAIL antibody [75411.11] (ab10516) from Abcam (http://www.abcam.com/TRAIL-antibody-75411-11-ab10516.html).

As explained above, TRAIL-R2-Fc fusion proteins suitable for use in the present invention is described in WO2015001345. Thus the invention may use an agent which is a fusion protein comprising an extracellular domain of a TRAIL-R, preferably of TRAIL-R2, or a portion thereof, fused to a portion of a human antibody, preferably an Fc domain, or a portion thereof, with or without the antibody hinge region, or a portion thereof.

The invention may utilise an agent that binds to TRAIL-R2, e.g. an antibody, or fragment thereof, that binds specifically to TRAIL-R2, neutralising its activity.

The invention may utilise an agent that binds to TRAIL-R1, e.g. an antibody, or fragment thereof, that binds specifically to TRAIL-R1, neutralising its activity.

The invention may utilise an agent that binds to TRAIL-R1 and TRAIL-R2, e.g. an antibody, or fragment thereof, that binds specifically to TRAIL-R1 and TRAIL-R2, neutralising their activity.

CD95L-binding protein consisting of the extracellular domain of human CD95 fused to the Fc region of human IgG1 has been used to block CD95 signalling^{12,13}. CD95L inhibitors include Apogenix's APG101 (Asunercept).

Emricasan is an orally active pan-caspase protease inhibitor suitable for use against Caspases.

Inhibition of TLR3 signalling can be achieved by small molecules that act as direct, competitive and high affinity inhibitors of dsRNA binding to TLR3¹⁴.

Like TLR3, TLR4 is known to be able to induce cell death. The ligand for TLR4 is LPS (lipopolysaccharide). Gao et al (2017) discuss the use of various TLR inhibitors/antagonists which target TLR signals to treat (amongst others) inflammatory disorders.

Ponatinib and pazopanib are known MLKL inhibitors. Kongensin A and Celastrol are known RIPK3 inhibitors.

In one embodiment of the invention the agents comprise a combination of three agents:
a TNF inhibitor (e.g. Enbrel, Humira, or Remicade),
an inhibitor of CD95L (e.g. Asunercept) and
an inhibitor of TRAIL (e.g. TRAIL-R2-Fc)

In a further embodiment the aforementioned combination is combined with an inhibitor of the kinase activity of RIPK1.

### Companion Diagnostics

The present invention provides for patient selection e.g. an individual suffering a disease has proved refractory to treatment with a TNF inhibitor or TNF inhibitors.

The invention may comprise screening patients for overexpression of one, more, or all of the combination of receptors, ligands or targets, the combined neutralisation of which the present therapeutic methods are based on. For example TNF, LT-α, TRAIL and CD95L, etc.

This may be done in order to select or reject patients for treatment with the agents described herein (“companion diagnostics”). For example the method may comprise assessing whether the target is expressed above a certain threshold, and treating the patient with the combination treatment described herein if the threshold is exceeded.

For companion diagnostics, a typical sample comprising nucleic acid or proteins is used, which may be selected from the group consisting of a tissue, a biopsy probe, cell lysate, cell culture, cell line, organ, organelle, biological fluid, blood sample, urine sample, skin sample, and the like.

For example, blood or biopsy could be withdrawn from a patient upon diagnosis of an inflammatory or an inflammation-associated disease and screened for the relevant targets.

Methods of assessing gene expression via RNA or protein levels are known in the art. RNA levels can be measured by any methods known to those of skill in the art such as, for example, differential screening, subtractive hybridization, differential display, and microarrays. A variety of protocols for detecting and measuring the expression of proteins, using either polyclonal or monoclonal antibodies specific for the proteins, are known in the art. Examples include Western blotting, enzyme-linked immunosorbent assay (ELISA), radioimmunoassay (RIA), and fluorescence activated cell sorting (FACS).

Preferred examples include histopathological analysis, immunohistochemistry (IHC), in situ hybridisation, RNAscope or flow cytometry (FACS). The use or real-time quantitative PCR has been used for many years to quantify gene expression (see e.g. Giulietti, Annapaula, et al. Methods 25.4 (2001): 386-401).

Furthermore assays for many targets are commercially available e.g. from Abcam (Human FAS Ligand ELISA Kit; Human TRAIL ELISA Kit etc.), R&D Systems (Human TNF-alpha Quantikine ELISA Kit) etc.

The invention may alternatively or additionally comprise screening patients for cell death markers.

For example, blood or biopsy could be withdrawn from a patient upon diagnosis of an inflammatory or an inflammation-associated disease and screened for cell death markers such as cleaved caspase-3 or TUNEL positivity. Alternatively, a patient that has been treated with for example an anti-inflammatory drug or with anti-TNF and has been refractory to such treatments could also be subjected to this screening. If a patient proves positive for cell death markers, they may be selected for treatment according to the present invention.

A commercially available diagnostic kit for detecting cell death is, for example, the ApopTag Red In Situ Apoptosis Detection kit by Merck Millipore, for detecting of DNA strand breaks, as a marker of cell death. This kit is particularly effective with formalin-fixed tissues.

Another commercially available diagnostic technique for detection of cell death is the in situ detection of cleaved (i.e. activated) caspase-3 (Cell Signalling, 9664)¹¹. Alternatively, cell death can be detected by CellTiter-Glo Luminescent Cell Viability Assay kit (Promega) or by FACS analysis using DNA-intercalating agents or antibodies⁹.

The present invention further provides the use of such cell death detection tools as companion diagnostic to this invention.

The present invention further includes the use of such kits for determining likelihood of effectiveness of treatment by the combinations of agents described herein in the subject.

### Inflammatory Disease

“Inflammatory disease” includes inflammation and inflammation-associated diseases including autoimmunity and cancer.

Examples include several inflammatory and autoimmune diseases including inflammatory bowel disease (including Crohn's disease and ulcerative colitis), psoriasis, retinal detachment (and degeneration), retinitis pigmentosa, macular degeneration, pancreatitis, atopic dermatitis, arthritis (including rheumatoid arthritis, spondyloarthritis, gout, systemic onset juvenile idiopathic arthritis (SoJIA), psoriatic arthritis), systemic lupus erythematosus (SLE), Sjogren's syndrome, systemic scleroderma, anti-phospholipid syndrome (APS), vasculitis, osteoarthritis, liver damage/diseases (non-alcohol steatohepatitis, alcohol steatohepatitis, autoimmune hepatitis, autoimmune hepatobiliary diseases, primary sclerosing cholangitis (PSC), acetaminophen toxicity, hepatotoxicity), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection, ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), neonatal hypoxic brain injury, allergic diseases (including asthma and atopic dermatitis), burns (burn injury, burn shock), multiple sclerosis, type I diabetes, Wegener's granulomatosis, pulmonary sarcoidosis, Behcet's disease, interleukin-1 converting enzyme (ICE, also known as caspase-1)-associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-κB essential modulator gene (also known as IKK-gamma or IKKG)), particularly, NEMO-deficiency syndrome, HOIL-1 mutations ((also known as RBCK1) heme-oxidized IRP2 ubiquitin ligase-1 deficiency), HOIP mutations ((also known as RNF31) HOIL-1-Interacting Protein), XIAP mutations ((also known as BIRC4) X-Linked Inhibitor Of Apoptosis), OTULIN mutations ((also known as FAM105B) OTU Deubiquitinase With Linear Linkage Specificity), CYLD mutations (Cylindromatosis), SPATA2 mutations (Spermatogenesis Associated 2), A20 mutations (also known as TNFAIP3), FADD mutations (Fas Associated Via Death Domain), Caspase-8 mutations, or hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, and rejection of transplant organs, tissues and cells and any type of inflammation-associated cancer.

In one embodiment the inflammatory disease caused by any of HOIL-1, HOIP or OTULIN deficiencies e.g. mutations (see e.g. Krenn, Martin, et al. “Mutations outside the N-terminal part of RBCK1 may cause polyglucosan body myopathy with immunological dysfunction: expanding the genotype-phenotype spectrum.” Journal of neurology (2017): 1-8; Boisson, Bertrand, et al. “Human HOIP and LUBAC deficiency underlies autoinflammation, immunodeficiency, amylopectinosis, and lymphangiectasia.” Journal of Experimental Medicine 212.6 (2015): 939-951.)

In one embodiment the inflammatory disease is selected from the list consisting of: an auto-immune disease optionally selected from multiple sclerosis (MS), amyotrophic lateral sclerosis (ALS); a neuro-inflammatory disease, which is optionally muscular dystrophy; a neuro-degenerative disease optionally selected from Parkinson's Disease, Alzheimer's Disease, and Huntington's Disease; an ischaemic disease optionally selected from ischaemic diseases of the heart, the kidney or the brain; sepsis.

Preferred target diseases are those shown in Table 3 which lists diseases in which TNF inhibition is believed to be of benefit, including those in which certain patients have not responded successfully (e.g. patients that do not respond to the initial treatment or lose response over time).

TABLE 3
selected diseases in which TNF inhibition
is believed to be of benefit

| Disease | References |
|---|---|
| rheumatoid arthritis (RA) | (Cho and Feldman, 2015) |
| Psoriasis | (Chaudhari et al., 2001) |
| psoriatic arthritis (PsA) | (Mease, 2002) |
| inflammatory bowel disease (IBD) | (Roda et al., 2016) |
| Crohn disease (CD) | (Hanauer et al., 2002) |
| ulcerative colitis (UC) | (Fausel and Afzali, 2015) |
| ankylosing spondylitis (AS) | (Liu et al., 2016) |
| juvenile idiopathic arthritis (JIA) | (Kearsley-Fleet et al., 2016) |
| hidradenitis suppurativa (HS) | (Lee and Eisen, 2015) |
| amyloidosis | (Fernandez-Nebro et al., 2010) |
| systemic lupus erythematosus (SLE) | (Stohl, 2013) |
| Behcet's disease | (Croft and Siegel, 2017) |
| asthma | Croft (Croft and Siegel, 2017) |
| multiple sclerosis | Arnason (1999) |
| Wegener's granulomatosis (WG) | (Cessak et al., 2014) |
| Sarcoidosis | (Cessak et al., 2014) |
| osteoarthritis | (Cessak et al., 2014) |
| Alzheimer's disease | (Cessak et al., 2014) |
| Kawasaki disease | (Cessak et al., 2014) |
| COPD | (Cessak et al., 2014) |
| pneumonia | (Cessak et al., 2014) |
| Sjogren's syndrome | (Meijer et al., 2007) |
| Parkinson disease | (Tweedie et al., 2007) |
| OTULIN-related autoinflammatory | (Damgaard et al., 2016) |
| syndrome (ORAS) | |
| HOIL-1 deficiency-related | (Boisson et al., 2012) |
| immunodeficiency | |

In one most preferred embodiment, the inflammatory disease is selected from the list consisting of rheumatoid arthritis (RA); psoriasis; inflammatory bowel disease (IBD).

In another embodiment the inflammatory disease is a cancer, and the method further comprises administering to the individual one or more additional agents for treating said cancer or performing radiotherapy on said individual. Optionally, the one or more additional agents for treating said cancer are selected from the lists consisting of chemotherapeutics; immune checkpoint inhibitors optionally selected from anti-PD-1/L1 and/or anti-CTLA-4 antibodies; cell-based therapies optionally selected from such as transgenic chimaeric antigen receptor (CAR)- or T cell receptor (TCR)-expressing T cells.

### Combination Therapies

The methods or treatments of the present invention are combination therapies utilising at least 3 agents.

The agents may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., a compound as described here, plus one or more other agents) may be formulated together in a single dosage form, or alternatively, the individual agents may be formulated separately and presented together in the form of a kit, optionally with instructions for their use.

In another embodiment the combinatorial therapies in this invention may be administered in combination with at least one other therapeutically active agent, wherein the other therapeutically active agent is selected from a thrombolytic agent, a tissue plasminogen activator, an anticoagulant, a platelet aggregation inhibitor, an antimicrobial agent (an antibiotic, a broad-spectrum antibiotic, a β-lactam, an antimycobacterial agent, a bactericidal antibiotic, anti-MRSA therapy), a long acting beta agonist, a combination of an inhaled corticosteroid and a long acting beta agonist, a short acting beta agonist, a leukotriene modifier, an anti-IgE, a methylxanthine bronchodilator, a mast cell inhibitor, a protein tyrosine kinase inhibitor, a CRTH2/D prostanoid receptor antagonist, an epinephrine inhalation aerosol, a phosphodiesterase inhibitor, a combination of a phosphodiesterase-3 inhibitor and a phosphodiesterase-4 inhibitor, a long-acting inhaled anticholinergic, a muscarinic antagonist, a long-acting muscarinic antagonist, a low dose steroid, an inhaled corticosteroid, an oral corticosteroid, a topical corticosteroid, anti-thymocyte globulin, thalidomide, chlorambucil, a calcium channel blocker, a topical emollient, an ACE inhibitor, a serotonin reuptake inhibitor, an endothelin-1 receptor inhibitor, an anti-fibrotic agent, a proton-pump inhibitor, a cystic fibrosis transmembrane conductance regulator potentiator, a mucolytic agent, pancreatic enzymes, a bronchodilator, an ophthalmic intravitreal injection, an anti-vascular endothelial growth factor inhibitor, a ciliary neurotrophic growth factor agent, a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, inactivated influenza vaccine, a ciliary neurotrophic growth factor, a gene transfer agent, a topical immunomodulator, calcineurin inhibitor, an interferon gamma, an antihistamine, a monoclonal antibody, a polyclonal anti-T-cell antibody, an anti-thymocyte gamma globulin-equine antibody, an anti-thymocyte globulin-rabbit antibody, an anti-CD40 antagonist, a JAK inhibitor, and an anti-TCR murine mAb.

Exemplary other therapeutically active agents include heparin, Coumadin, clopidrogel, dipyridamole, ticlopidine HCL, eptifibatide, aspirin, vacomycin, cefeprime, a combination of piperacillin and tazobactam, imipenem, meropenem, doripenem, ciprofloxacin, levofloxacin, ofloxacin, moxifloxacin, hydrocortisone, vedolizumab, alicaforsen, remestemcel-L, ixekizumab, tildrakizumab, secukinumab, chlorhexidine, doxycycline, minocycline, fluticasone (fluticasone proprionate, fluticasone furoate), beclomethasone dipropionate, budesonide, trimcinolone acetonide, flunisolide, mometasone fuorate, ciclesonide, arformoterol tartrate, formoterol fumarate, salmeterol xinafoate, albuterol (albuterol sulfate), levalbuterol tartrate, ipratropium bromide, montelukast sodium, zafirlukast, zileuton, omalizumab, theophylline, cromulyn sodium, nedocromil sodium, masitinib, AMG 853, indacaterol, E004, reslizumab, salbutamol, tiotropium bromide, VR506, lebrikizumab, RPL554, afibercept, umeclidinium, indacterol maleate, aclidinium bromide, roflumilast, SCH527123, glycopyrronium bromide, olodaterol, a combination of fluticasone furoate and vilanterol vilanterol, a combination of fluticasone propionate and salmeterol, a combination of fluticasone furoate and fluticasone proprionate, a combination of fluticasone propionate and eformoterol fumarate dihydrate, a combination of formoterol and budesonide, a combination of beclomethasone dipropionate and formoterol, a combination of mometasone furoate and formoterol fumarate dihydrate, a combination of umeclidinium and vilanterol, a combination of ipratropium bromide and albuterol sulfate, a combination of glycopyrronium bromide and indacaterol maleate, a combination of glycopyrrolate and formoterol fumarate, a combination of aclidinium and formoterol, isoniazid, ehambutol, rifampin, pyrazinamide, rifabutin, rifapentine, capreomycin, levofloxacin, moxifloxicin, ofloxacin, ehionamide, cycloserine, kanamycin, streptomycin, viomycin, bedaquiline fumarate, PNU-100480, delamanid, imatinib, ARG201, tocilizumab, muromonab-CD3, basiliximab, daclizumab, rituximab, prednisolone, anti-thymocyte globulin, FK506 (tacrolimus), methotrexate, cyclosporine, sirolimus, everolimus, mycophenolate sodium, mycophenolate mofetil, cyclophosphamide, azathioprine, thalidomide, chlorambucil, nifedipine, nicardipine, nitroglycerin, lisinopril, diltaizem, fluoxetine, bosentan, epoprostenol, colchicine, para-aminobenzoic acid, dimethyl sulfoxide, D-penicillamine, interferon alpha, interferon gamma (INF-g)), omeprazole, metoclopramide, lansoprazole, esomeprazole, pantoprazole, rabeprazole, imatinib, belimumab, ARG201, tocilizumab, ivacftor, dornase alpha, pancrelipase, tobramycin, aztreonam, colistimethate sodium, cefadroxil monohydrate, cefazolin, cephalexin, cefazolin, moxifloxacin, levofloxacin, gemifloxacin, azithromycin, gentamicin, ceftazidime, a combination of trimethoprim and sulfamethoxazole, chloramphenicol, a combination of ivacftor and lumacaftor, ataluren, NT-501-CNTF, a gene transfer agent encoding myosin VIIA (MY07A), ranibizumab, pegaptanib sodium, NT501, humanized sphingomab, bevacizumab, oseltamivir, zanamivir, rimantadine, amantadine, nafcillin, sulfamethoxazolem, trimethoprim, sulfasalazine, acetyl sulfisoxazole, vancomycin, muromonab-CD3, ASKP-1240, ASP015K, TOL101, pimecrolimus, hydrocortizone, betamethasone, flurandrenolide, triamcinolone, fluocinonide, clobetasol, hydrocortisone, methylprednisolone, prednisolone, a recombinant synthetic type I interferon, interferon alpha-2a, interferon alpha-2b, hydroxyzine, diphenhydramine, flucloxacillin, dicloxacillin, and erythromycin.

In another embodiment the combinatorial therapies in this invention may be administered in combination with at least one other therapeutically active agent—for example may be administered in combination with other anti-inflammatory agents for any of the indications above, including oral or topical corticosteroids, 5-aminosalicyclic acid and mesalamine preparations, hydroxyeloroquine, thiopurines, methotrexate, cyclophosphamide, cyclosporine, calcineurin inhibitors, mycophenolic acid, mTOR inhibitors, JAK inhibitors, Syk inhibitors, anti-inflammatory biologic agents, including anti-IL-6 biologics, anti-IL-1 agents (including anti-IL1β and anti-IL-1α biologics), anti-I-17 biologics, anti-CD22, anti-integrin agents, anti-IFNα, anti-CD20 or CD4 biologics and other cytokine inhibitors or biologics to T-cell or B-cell receptors or interleukins.

Methods described herein may comprise administering to a subject in need of such treatment a “therapeutically effective” amount of agents that decrease the biological activity of the ligands or receptor. Agents capable of decreasing the biological activity may achieve their effect by a number of means. For instance, such an agent may be one which (by way of non-limiting example) decreases the expression of the receptor; increases receptor desensitisation or receptor breakdown; reduces interaction between ligands their endogenous receptors; reduces receptor mediated intracellular signalling; competes with endogenous receptors for ligand binding; binds to the receptors to block ligand binding; or binds to the ligand preventing interaction with its receptors.

It is preferred that the agents directly interacts with the receptor or ligand.

In one preferred embodiment the agent binds to and blocks activity of the receptor or ligand, or it binds and blocks the endogenous ligand/receptor complex from forming properly so that it can no longer engage in the intracellular signalling.

An example of a biotherapeutic drug that can interact with such targets is an antibody, for example a human or humanised antibody. The antibodies in this invention may be monoclonal, polyclonal, chimeric, single chain antibodies or functional antibody fragments.

Another example of a biotherapeutic drug is a soluble receptor protein, e.g. a soluble receptor-Fc fusion protein which contains the extracellular portion of the receptor, or at least a portion thereof that is capable of binding to the ligand in a manner that (the receptor-stimulating activity of) the respective ligand in question is inhibited.

For brevity embodiments below may be described by way of non-limiting example with respect TRAIL, or a TRAIL-R such as TRAIL-R1 or TRAIL-R2. Nevertheless it will be appreciated that all such discussion applies mutatis mutandis to any other TRAIL-R for example TRAIL-R1, TRAIL-R3, or TRAIL-R4. It will also be appreciated that all such discussion applies mutatis mutandis to other ligands and their respective receptors described herein.

### Antibodies

For the production of antibodies according to the invention, various host species may be immunised by injection with the above mentioned proteins to be targeted or any fragments of the two proteins which are immunogenic.

For example antibodies to neutralise TRAIL activity may be raised against full length human TRAIL, sequences.

An appropriate adjuvant will be chosen depending on the host species in order to increase an immune response. Preferentially, peptides, fragments or oligopeptides used to induce an antibody response against them will contain at least five, but preferably ten amino acids. Monoclonal antibodies against the two proteins may be produced using any technique that provides for the production of antibody molecules or recombinant and non-recombinant functional fragments of these antibodies by continuous cell lines in culture. These include, but are not limited to, the hybridoma technique and the human B-cell hybridoma technique. In addition, techniques developed for the production of chimeric antibodies, e.g. recombinant antibodies can be used. Resulting antibodies may be used with or without modifications such as labelling, recombinant joining of antibody stretches or with molecules functioning as reporters. Modifications can be covalent and/or non-covalent.

Many different immune- and non-immunoassays may be used for screening to identify antibodies with the desired specificity. Various protocols for competitive binding and immunoradiometric assays using either polyclonal or monoclonal antibodies with already established specificity are well known in the field. These immunoassays typically involve measuring complex formation between the receptor or ligand and their specific antibodies. A “Sandwich”, i.e. two-sided, monoclonal-based immunoassay is preferred that comprises monoclonal antibodies against two non-interfering protein epitopes, but a competitive binding assay may also be used.

More specifically, it is preferred that the antibody is a γ-immunoglobulin (IgG).

It will be appreciated that the variable region of an antibody defines the specificity of the antibody and as such this region should be conserved in functional derivatives of the antibody according to the invention. The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies according to the invention may have very different C_{H} and C_{L} domains. Furthermore preferred antibody functional derivatives may comprise the Variable domains without a C-domain (e.g. scFV antibodies).

An antibody derivative may have 75% sequence identity, more preferably 90% sequence identity and most preferably has at least 95% sequence identity to a monoclonal antibody or specific antibody in a polyclonal mix. It will be appreciated that most sequence variation may occur in the framework regions (FRs) whereas the sequence of the CDRs of the antibodies, and functional derivatives thereof, is most conserved.

A number of preferred embodiments of the invention relate to molecules with both Variable and Constant domains. However it will be appreciated that antibody fragments (e.g. scFV antibodies) are also encompassed by the invention that comprise essentially the Variable region of an antibody without any Constant region.

Antibodies generated in one species are known to have several serious drawbacks when used to treat a different species. For instance when murine antibodies are used in humans they tend to have a short circulating half-life in serum and are recognised as foreign proteins by the patient being treated. This leads to the development of an unwanted human anti-mouse (or rat) antibody response. This is particularly troublesome when frequent administrations of the antibody is required as it can enhance the clearance thereof, block its therapeutic effect, and induce hypersensitivity reactions. Accordingly preferred antibodies (if of non-human source) for use in human therapy are humanised.

Monoclonal antibodies are generated by the hybridoma technique which usually involves the generation of non-human mAbs. The technique enables rodent monoclonal antibodies with almost any specificity to be produced. Accordingly preferred embodiments of the invention may use such a technique to develop monoclonal antibodies against the TRAIL receptors. Although such antibodies are useful therapeutically, it will be appreciated that such antibodies are not ideal therapeutic agents in humans (as suggested above). Ideally, human monoclonal antibodies would be the preferred choice for therapeutic applications. However, the generation of human mAbs using conventional cell fusion techniques has not to date been very successful. The problem of humanisation may be at least partly addressed by engineering antibodies that use V region sequences from non-human (e.g. rodent) mAbs and C region (and ideally FRs from V region) sequences from human antibodies. The resulting ‘engineered’ mAbs are less immunogenic in humans than the rodent mAbs from which they were derived and so are better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric antibodies are superior to non-human monoclonal antibodies in that their ability to activate effector functions can be tailored for a specific therapeutic application, and the anti-globulin response they induce is reduced.

Such chimaeric molecules are preferred agents for treating disease according to the present invention. RT-PCR may be used to isolate the V_{H} and V_{L} genes from preferred mAbs, cloned and used to construct a chimaeric version of the mAb possessing human domains.

Further humanisation of antibodies may involve CDR-grafting or reshaping of antibodies. Such antibodies are produced by transplanting the heavy and light chain CDRs of a rodent mAb (which form the antibody's antigen binding site) into the corresponding framework regions of a human antibody.

### Fragments or Fusion Proteins

Agents as described herein may be based on portions (e.g. soluble fragments) of receptors, optionally fused to heterologous protein domains or combined with non-protein moieties.

By way of non-limiting example, a TRAIL inhibitor comprises the extracellular domain of TRAIL-R1, TRAIL-R2, TRAIL-R3, TRAIL-R4 or OPG, preferentially that of TRAIL-R2, or a ligand-binding portion thereof, or the extracellular domain of the mature TRAIL-R2 sequence according to Walczak et al. (Walczak, H., Degli-Esposti, M. A., Johnson, R. S., Smolak, P. J., Waugh, J. Y., Boiani, N., Timour, M. S., Gerhart, M. J., Schooley, K. A., Smith, C. A., et al. (1997). TRAIL-R2: a novel apoptosis-mediating receptor for TRAIL. The EMBO journal 16, 5386-5397) and a patent by C. T. Rauch and H. Walczak (U.S. Pat. No. 6,569,642 B1), which is specifically incorporated herein by reference, which may be fused to a heterologous polypeptide domain, particularly an Fc portion of an immunoglobulin molecule, including or not the hinge region or part thereof, e.g. from a human IgG molecule, preferably an Fc region of human IgG1, IgG2, IgG3 or human IgG4 with or without the hinge region or a part thereof.

The way the two fully human protein parts are fused can be done in a manner that reduces the immunogenicity potential of the resulting fusion protein as described in Walczak (WO/2004/085478; PCT/EP2004/003239: “Improved Fc fusion proteins”).

Because there are two splice forms of TRAIL-R2 expressed and the splicing affects the extracellular domain of TRAIL-R2 (Screaton, G. R., Mongkolsapaya, J., Xu, X. N., Cowper, A. E., McMichael, A. J., and Bell, J. I. (1997). TRICK2, a new alternatively spliced receptor that transduces the cytotoxic signal from TRAIL. Current biology: CB 7, 693-696.), at least two extracellular domains of TRAIL-R2 with differing amino acid sequences are known. In one embodiment, the TRAIL-binding portion of the extracellular domain of TRAIL-R2 can come from either one of these two when constructing TRAIL-inhibiting TRAIL-R2 fusion proteins.

TRAIL-R2-Fc fusions suitable for use in the present is described in WO2015001345, the contents of which, particularly in respect of TRAIL-R2-Fc fusions, is explicitly incorporated herein by cross reference. The TRAIL-R2-Fc polypeptide from WO2015001345 is set out below. The TRAIL-R2 portion is underlined. The Fc portion is depicted in bold. Note that there is a one amino acid overlap between TRAIL-R2 portion and the human IgG1 FC portion. The leader peptide is depicted in italics. The mature protein starts with the sequence ITQQDLA. When produced recombinantly, the exact position of the N terminus can vary by a few amino acids; that means the mature protein can be, e.g. one to five amino acids shorter or longer.

MEQRGQNAPAASGARKRHGPGPREARGARPGPRVPKTLVLVVAAVLLLV
SAESALITQQDLAPQQRAAPQQKRSSPSEGLCPPGHHISEDGRDCISCK
YGQDYSTHWNDLLFCLRCTRCDSGEVELSPCTTTRNTVCQCEEGTFREE
DSPEMCRKCRTGCPRGMVKVGDCTPWSDIECVHKESGTKHSGEVPAVEE
TVTSSPGTPASCDKTHTCPPCPAPELLGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSF
FLYSKLTVDKSRWQQGNVFSCSVMHEALHNHYTQKSLSLSPGK

TRAIL-R fusion proteins that bind to and neutralise TRAIL activity may be produced using any technique that provides for the production of recombinant and non-recombinant full length or functional fragments of these proteins by continuous cell lines in culture.

As described below, resulting proteins may be used with or without modifications such as labelling, recombinant joining of antibody stretches or with molecules functioning as reporters. Modifications can be covalent and/or non-covalent.

### Peptide Agents

It will be appreciated that peptide or protein agents used or provided according to the invention may be derivatives of native or original sequences, and thus include derivatives that increase the effectiveness or half-life of the agent in vivo. Examples of derivatives capable of increasing the half-life of polypeptides according to the invention include peptoid derivatives, D-amino acid derivatives and peptide-peptoid hybrids.

Proteins and peptide agents according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of proteins and peptides according to the invention may be readily designed from knowledge of the structure of the receptor according to the first aspect of the invention or an agent according to the fourth, fifth or sixth aspect of the invention. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

### Nucleic Acids

In a further embodiment of the present invention the agent or inhibitor is a nucleic acid effector molecule.

The nucleic acid effector molecule may be DNA, RNA (including siRNA, miRNA and shRNA), PNA or a DNA-RNA-hybrid molecule. These may be specifically directed towards down-regulation of TRAIL or TRAIL-R sequences (see e.g. Example 5). siRNA forms part of a gene silencing mechanism, known as RNA interference (RNAi) which results in the sequence-specific destruction of mRNAs and enables a targeted knockout of gene expression. siRNA used in gene silencing may comprise double stranded RNA of 21 nucleotides length, typically with a 2-nucleotide overhang at each 3′ end. Alternatively, short hairpin RNAs (shRNAs) using sense and antisense sequences connected by a hairpin loop may be used. Both siRNAs and shRNAs can be either chemically synthesized and introduced into cells for transient RNAi or expressed endogenously from a promoter for long-term inhibition of gene expression. siRNA molecules for use as an agent according to the invention may comprise either double stranded RNA of 10-50 nucleotides. Preferably, siRNAs for use as an agent according to the invention comprise 18-30 nucleotides. More preferably, siRNAs for use as an agent according to the invention comprise 21-25 nucleotides. And most preferably, siRNAs for use as an agent according to the invention comprise 21 nucleotides. It will be appreciated that siRNAs will need to be based upon the sequences according to the second aspect of the invention. Preferred double stranded siRNA molecules comprise a sense strand of 21-25 contiguous nucleotides from a sequence of the TRAIL or its receptors bound to the complementary antisense strand. Alternatively, shRNAs using sense and antisense sequences may be used as an agent according to the invention. Preferably, shRNAs using sense and antisense sequences that may be employed as an agent according to the invention comprise 20-100 nucleotides.

In other embodiments the nucleic acid may encode other agents of the invention for example the fusion proteins described.

The nucleic acid may be single or double-stranded. The nucleic acid effector molecule may be delivered directly as a drug (this could be “naked” or e.g. in liposomes) it may be expressed from a retrovirus, adenovirus, herpes or vaccinia virus or bacterial plasmids for delivery of nucleotide sequences to the targeted organ, tissue or cell population.

These constructs may be used to introduce untranslatable sense or antisense sequences into a cell.

Without integration into the DNA, these vectors may continue to produce RNA molecules until degradation by cellular nucleases. Vector systems may result in transient expression for one month or more with a non-replicating vector and longer if appropriate replication elements are part of the vector system.

Thus, as is well known in the art, recombinant vectors may include other functional elements. For instance, recombinant vectors can be designed such that the vector will autonomously replicate in the cell. In this case, elements which induce DNA replication may be required in the recombinant vector. Alternatively, the recombinant vector may be designed such that the vector and nucleic acid molecule integrates into the genome of a cell. In this case DNA sequences which favour targeted integration (e.g. by homologous recombination) are desirable. Recombinant vectors may also have DNA coding for genes that may be used as selectable markers in the cloning process. The recombinant vector may also further comprise a promoter or regulator to control expression of the nucleic acid as required.

### Variants

Wherever amino acid and nucleic acid sequences are discussed herein (for example in respect of coding fusion proteins or other agents), it will be appreciated by the skilled technician that functional derivatives of the amino acid, and nucleic acid sequences, disclosed herein, are also envisaged—such derivatives may have a sequence which has at least 30%, preferably 40%, more preferably 50%, and even more preferably, 60% sequence identity with the amino acid/polypeptide/nucleic acid sequences of any of the sequences referred to herein. An amino acid/polypeptide/nucleic acid sequence with a greater identity than preferably 65%, more preferably 75%, even more preferably 85%, and even more preferably 90% to any of the sequences referred to is also envisaged. Preferably, the amino acid/polypeptide/nucleic acid sequence has 92% identity, even more preferably 95% identity, even more preferably 97% identity, even more preferably 98% identity and, most preferably, 99% identity with any of the referred to sequences.

Calculation of percentage identities between different amino acid/polypeptide/nucleic acid sequences may be carried out as follows. A multiple alignment is first generated by the ClustalX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Alternatively, a substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention.

Due to the degeneracy of the genetic code, it is clear that any nucleic acid sequence could be varied or changed without substantially affecting the sequence of the receptor protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The accurate alignment of protein or DNA has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor programs is Align (http://www.gwdg.de/˜dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYLIP.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

### Compositions, Dosages and Regimens

The agents utilised in the present invention (e.g. which binds TNF/LT-α, TRAIL, CD95L or TNFR1, TRAIL-Rs, CD95, TLR3, TLR4, Caspase-8, RIPK3, MLKL or RIPK1 that neutralises cell death and inflammation triggered by TNF/LT-α/TNFR1, TRAIL/TRAIL-Rs, CD95L/CD95, dsRNA/TLR3, LPS/TLR4, RIPK1, Caspase-8, RIPK3 and MLKL) may be provided as a “pharmaceutical composition”.

Pharmaceutical compositions may be administered alone or in combination with at least one other agent, such as stabilising compounds, which may be administered in any sterile, biocompatible pharmaceutical carrier solution, including, but not limited to saline, buffered saline, dextrose and water. The compositions may be administered to patients alone or in combination with other agents, drugs or hormones. The pharmaceutical compositions detailed in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

Pharmaceutical compositions will generally comprise the agents in an effective amount to achieve the intended purpose.

The determination of an effective dose is well within the capability trained personnel. For any compounds, the therapeutically effective dose can be estimated initially either in cell culture assays, e.g., of cell lines or in animal models, usually but not exclusively mice. The animal model may also be used to determine the appropriate concentration range and route of administration. Based on such pilot experiments, useful doses and routes for administration in humans can be determined. A therapeutically effective dose refers to that amount of active ingredient, for example a nucleic acid or a protein of the invention or an antibody, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as LD50/ED50. Pharmaceutical compositions, which exhibit large therapeutic indices, are preferred. The dosage is preferably within a range of circulating concentrations that include the ED50 with little or no toxicity. The dosage varies within this range depending upon the dosage employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells and conditions as detailed above.

### General Statements

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress (prolonged survival), a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention can be much lower than was hitherto understood in the art.

The invention also embraces treatment as a prophylactic measure is also included and “treating” will be understood accordingly. Prophylactic treatment may utilise a “prophylactically effective amount,” which where used herein pertains to that amount of an agent which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

Wherever a method of treatment employing an agent is described herein, it will be appreciated that an agent (any one of the first, second, third agents) for use in that method is also described, as is an agent (any one of the first, second, third agents) for use in the manufacture of a medicament for treating the relevant inflammatory disease. Also described is any one of the first, second, third agents for use in methods of enhancing the activity of the other two agents.

Wherever a composition is described herein, it will be appreciated that the same composition for use in the therapeutic methods (including prophylactic methods) described herein is also envisaged, as is the composition for use in the manufacture of a medicament for treating the relevant inflammatory disease.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

### FIGURES

FIG. 1. Deletion of HOIP in keratinocytes results in TNFR1-dependent postnatal lethality and TNFR1-independent lethal dermatitis at a later age. a, d, g, Representative images of mice with the indicated genotypes, (n=10 mice per genotype) (a, g). Animals were treated with vehicle or 4-OHT every other day for a total of 4 doses (n=3 mice per genotype) (d). b, e, h, Representative images of skin sections stained with H&E or with the indicated antibodies from mice with the indicated genotypes (n=3 mice per genotype). Arrows: pyknotic nuclei, stars: immune cell infiltrates, arrowhead: parakeratosis and black bar: hyperkeratosis. Nuclei were stained with DAPI (blue). White dashed lines separate the epidermis (above) from the dermis (below). Scale bars, 50 μm. c, f, i, Representative images of skin sections double stained with TUNEL (red) and CC3 antibody (green) in mice with the indicated genotypes (top panels). Nuclei were stained with DAPI (blue). White dashed lines separate the epidermis (above) from the dermis (below). Scale bars, 50 μm. Quantification of TUNEL and CC3 positive cells in skin sections from mice with the indicated genotypes (bottom panels) (n=3 mice per genotype). Error bars represent mean values±standard error of mean (s.e.m). *P≤0.05, ***P≤0.001. CC3: cleaved Caspase-3. Control mice represent a pool of Hoip^{fl/fl};K14-Cre− and Hoip^{fl/wt};K14-Cre+ (a-c) or Tnfr1^{KO};Hoip^{fl/fl};K14-Cre− and Tnfr1^{KO};Hoip^{fl/wt};K14-Cre+ mice (g-i).

FIG. 2. Loss of HOIL-1 causes TNFR1-dependent and TNFR1-independent lethal dermatitis. a, d, Representative images of mice with the indicated genotypes, (n=10 mice per genotype). b, e, Representative images of skin sections stained with H&E or with the indicated antibodies from mice with the indicated genotypes (n=3 mice per genotype). Arrows: pyknotic nuclei, stars: immune infiltrates, arrowhead: parakeratosis and black bar: hyperkeratosis. Nuclei were stained with DAPI (blue). White dashed lines separate the epidermis (above) from the dermis (below). Scale bars, 50 μm. c, f, Representative images of skin sections double stained with TUNEL (red) and CC3 antibody (green) in mice with the indicated genotypes (top panels). Nuclei were stained with DAPI (blue). White dashed lines separate the epidermis (above) from the dermis (below). Scale bars, 50 μm. Quantification of TUNEL and CC3 positive cells in skin sections from mice with the indicated genotypes (bottom panels) (n=3 mice per genotype). Error bars represent mean values±s.e.m. **P≤0.01, ***P≤0.001. CC3: cleaved Caspase-3. Control mice represent a pool of Hoil-1^{fl/fl};K14-Cre− and Hoil-1^{fl/wt};K14-Cre+ (a-c) or Tnfr1^{KO};Hoil-1^{fl/fl};K14-Cre− and Tnfr1^{KO};Hoil-1^{fl/wt};K14-Cre+ mice (d-f).

FIG. 3. Aberrant apoptosis drives lethal dermatitis in Hoip^{E-KO} and Hoil-1^{E-KO} mice. a, Quantification of TUNEL and CC3 positive cells in skin sections from mice with the indicated genotypes (n=3 mice per genotype). Error bars represent mean values±s.e.m. *P≤0.05, **P≤0.01, ***P≤0.001. b, Representative images of skin sections from mice with the indicated genotypes (n=4) stained with antibody against CD45 (red) at P0. Nuclei were stained with DAPI (blue). White dashed lines separate the epidermis (above) from the dermis (below). Scale bar, 50 μm. c, Flow cytometry analysis of immune cells in skin samples from mice with the indicated genotypes at the indicated postnatal days. Bar graphs represent the percentage of CD45 positive cells relative to the Forward and Side scatter profile (n=5 mice per genotype). Error bars represent mean values±s.e.m. **P≤0.01, ***P≤0.001, NS=not significant. d, FADD IP was performed in PMKs derived from control (+) or Hoip^{E-KO} (−) mice cultured in presence of ZVAD-fmk (representative blot of n=2 independent experiment). Lysates and IP were analysed by Western blotting for the indicated proteins. e, PMKs derived from Hoip^{E-KO} and control mice were cultured for four days in absence (NT: not-treated) or presence of the inhibitors Necrostatin-1s (N), ZVAD-fmk (Z) or RIPK3i. Cell viability (%) was measured by CellTiter-Glo assay on day four. Error bars represent mean values±s.e.m. (n=5). ***P≤0.001, NS=not significant. CC3: cleaved Caspase-3. f, Cell viability (%) measured by CellTiter-Glo assay of PMKs derived from adult mice with the indicated genotypes. Results are expressed as mean values±SEM (n=8 mice per genotype). NS=not significant. g, Representative images of mice with the indicated genotypes, (n=15 mice per genotype). h, Representative images of skin sections stained with H&E or with the indicated antibodies in mice with the indicated genotypes (n=3 mice per genotype). Arrows: pyknotic nuclei. Nuclei were stained with DAPI (blue). Scale bars, 50 μm. CC3: cleaved Caspase-3. i, Quantification of TUNEL and CC3 positive cells in skin sections from mice with the indicated genotypes (n=3 mice per genotype). Error bars represent mean values±s.e.m. *P≤0.05, ***P≤0.001, NS=not significant. j, Representative images of skin sections from mice with the indicated genotypes (n=4) stained with antibody against CD45 (red) at D70. Nuclei were stained with DAPI (blue). White dashed lines separate the epidermis (above) from the dermis (below). Scale bar, 50 μm. k, Kaplan Meier survival curve of mice with the indicated genotypes. Comparisons between Hoip^{E-KO} (n=10) and Mlkl^{KO};Hoip^{E-KO} (n=9) or Mlkl^{KO};Caspase-8^{KO};Hoip^{E-KO} (n=4) and, Hoil-1^{E-KO} (n=13) and Ripk3^{KO};Hoil-1^{E-KO} (n=8) or Caspase-8^{KO/WT};Hoil-1^{E-KO} (n=4) or Ripk3^{KO};Caspase-8^{KO/WT};Hoil-1^{E-KO} (n=11) or Ripk3^{KO};Caspase-8^{KO};Hoil-1^{E-KO} (n=15) mice were submitted for statistical analysis.**P≤0.01, ***P≤0.001, NS=not significant. Ripk3^{KO};Caspase-8^{KO};Hoil-1^{fl/wt}K14cre+ (n=13) and Mlkl^{KO};Caspase-8^{KO};Hoip^{fl-wt}K14cre+ (n=4) mice were used as controls. All mice with combined deficiency of Caspase-8 and MLKL or RIPK3 were culled when they developed severe lymphadenopathy and splenomegaly according to the regulations of the UK home office for animal welfare. Control mice represent a pool of Hoip^{fl/fl};K14-Cre− and Hoip^{fl/wt};K14-Cre+ or Hoil-1^{fl/fl};K14-Cre− and Hoil-1^{fl/wt};K14-Cre+ (a-e), Tnfr1^{KO};Hoip^{fl/fl};K14-Cre− and Tnfr1^{KO};Hoip^{fl/wt};K14-Cre+ mice (f), Ripk3^{KO};Hoil-1^{fl/fl};K14-Cre− and Ripk3^{KO};Hoil-1^{fl/wt};K14-Cre+ or Ripk3^{KO};Caspase-8^{KO};Hoil-1^{fl/fl};K14-Cre− and Ripk3^{KO};Caspase-8^{KO};Hoil-1^{fl/wt};K14-Cre+ mice (g-i).

FIG. 4. CD95L- and TRAIL-induced cell death drives TNFR1-independent dermatitis. a, PMKs derived from adult mice with the indicated genotypes were treated or not for 24 hours with TRAIL [50 ng/ml], CD95L [50 ng/ml] and Poly(I:C) [100 μg/ml]. Cell viability (%) was measured by CellTiter-Glo assay. Results are expressed as mean values±SEM. (n=7 mice per genotype). **p≤0.01, ***p≤0.001, NS=not significant. Control mice represent a pool of Tnfr1^{KO};Hoil-1^{fl/fl};K14-Cre− and Tnfr1^{KO};Hoil-1^{fl/wt};K14-Cre+ mice b, Representative images of mice with the indicated genotypes. c, Severity score of dermatitis was assessed at P70 in mice with the indicated genotypes. The total score was determined by evaluating the regions of the body affected by the lesions (black) and the character of the lesion (white). Tnfr1^{KO};Hoil-1^{E-KO} (n=6), Trail-r^{KO};Tnfr1^{KO};Hoil-1^{E-KO} (n=4), Tlr3^{KO};Tnfr1^{KO};Hoil-1^{E-KO} (n=4), Cd95^{E-DD}; Tnfr1^{KO}; Hoil-1^{E-KO} (n=12), Trail-r^{KO}; Tlr3^{KO};Tnfr1^{KO};Hoil-1^{E-KO} (n=20) and Cd95^{E-DD};Trail-r^{KO};Tnfr1^{KO};Hoil-1^{E-KO} (n=19), Mlkl^{KO};Tnfr1^{KO};Hoip^{E-KO} (n=13). d, Kaplan-Meier survival curve of mice with the indicated genotypes. Comparisons between Tnfr1^{KO};Hoil-1^{E-KO} or Tnfr1^{KO};Hoip^{E-KO} mice with mice with the indicated genotypes were submitted for statistical analysis *P≤0.05, ***P≤0.001; NS=not significant. Tnfr1^{KO};Hoil-1^{E-KO} (n=21), Trail-r^{KO};Tlr3^{KO};Tnfr1^{KO};Hoil-1^{E-KO} (n=19) and Cd95^{E-DD};Trail-r^{KO};Tnfr1^{KO};Hoil-1^{E-KO} (n=32), Mlkl^{KO};Tnfr1^{KO};Hoip^{E-KO} (n=17).

### EXTENDED DATA FIGURE LEGENDS

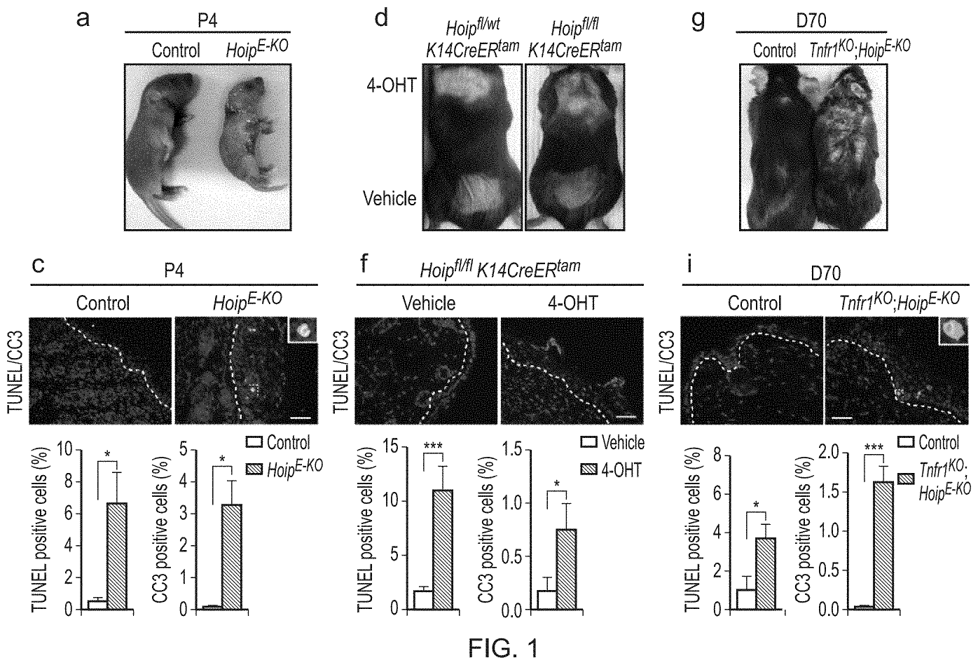

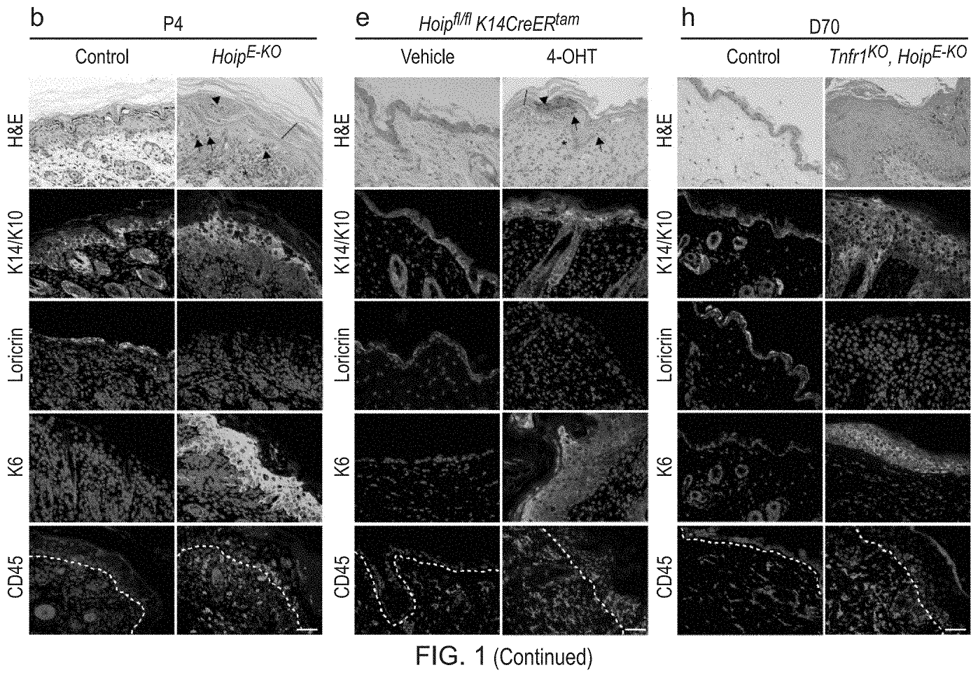

Extended Data FIG. 1: Generation and characterisation of HOIP deficiency in keratinocytes. a, PCR genotyping of DNA isolated from the ear punch of mice with the indicated genotypes. b, Western blot analysis of LUBAC components in PMKs derived from mice with the indicated genotypes. c, Representative images of skin sections stained with antibody against HOIP at P4. Scale bar, 50 μm. d, Endogenous TNFR1 complex I pull down was performed by FLAG IP in PMKs derived from control (+) or Hoip^{E-KO} (−)) mice cultured in presence of ZVAD-fmk and stimulated with FLAG-TNF. Lysates and IP were analysed by Western blotting for the indicated proteins. e, Western blot analysis of the indicated proteins in whole-cell lysates from PMKs derived from control (+) and Hoip^{E-KO} (−) mice following His-tagged TNF [100 ng/ml] stimulation for different time points (min). f, Epidermal thickness quantification of skin sections from mice with the indicated genotypes at P4 (n=4 per genotype). Error bars represent mean values±s.e.m. ***P≤0.001. g, Flow cytometry analysis of immune cells in skin samples from mice with the indicated genotypes at P4. Bar graphs show the percentage of CD45⁺, CD11b⁺GR-1⁺, CD11b⁺F4/80⁺ and CD19⁺, CD3⁺ cells relative to live and Side Scatter profile (n=5 per genotype). Error bars represent mean values±s.e.m. **P≤0.01, ***P≤0.001, NS=not significant. h, Representative images of skin sections of Hoip^{fl/wt}K14CreER^{tam} mice treated with vehicle or 4-OHT every other day for a total of 4 doses and stained with H&E or with the indicated antibodies (n=3 mice per genotype). Nuclei were stained with DAPI (blue). White dashed lines separate the epidermis (above) from the dermis (below). Scale bar, 50 μm. i, Epidermal thickness quantification of mice with the indicated genotypes and treated as in (h) (n=3 per genotype). Error bars represent mean values±s.e.m. *P≤0.05, NS=not significant. j, Quantification of CD45 staining in skin sections from mice with the indicated genotype treated as in h was performed by measuring overall fluorescence intensity using ImageJ. au=arbitrary units. k, Representative images of skin sections double stained with TUNEL (red) and CC3 antibody (green) in mice with the indicated genotypes (top panels). Nuclei were stained with DAPI (blue). White dashed lines separate the epidermis (above) from the dermis (below). Scale bars, 50 μm. Quantification of TUNEL and CC3 positive cells in skin sections from Hoip^{fl/wt}K14CreER^{tam} mice treated as indicated (bottom panel) (n=3 mice per genotype). CC3 was not detected (nd). Error bars represent mean values±s.e.m. NS=not significant. CC3: cleaved Caspase-3. Control mice represent a pool of Tnfr1^{KO}Hoip^{fl/fl};K14-Cre− and Tnfr1^{KO}Hoip^{fl/wt};K14-Cre+ mice (b) or Hoip^{fl/fl};K14-Cre− and Hoip^{fl/wt};K14-Cre+ mice (c, f, g).

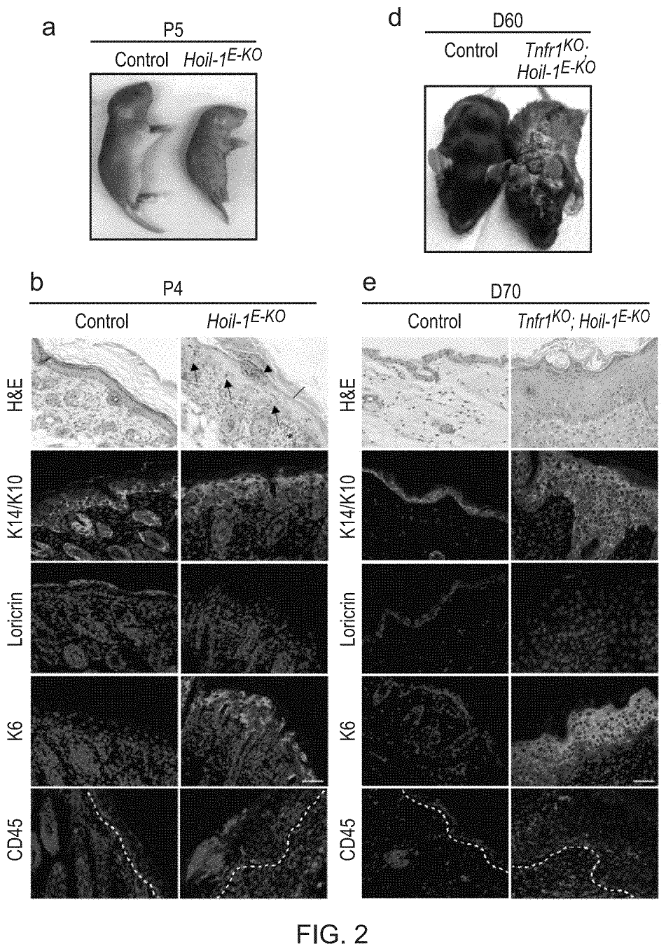

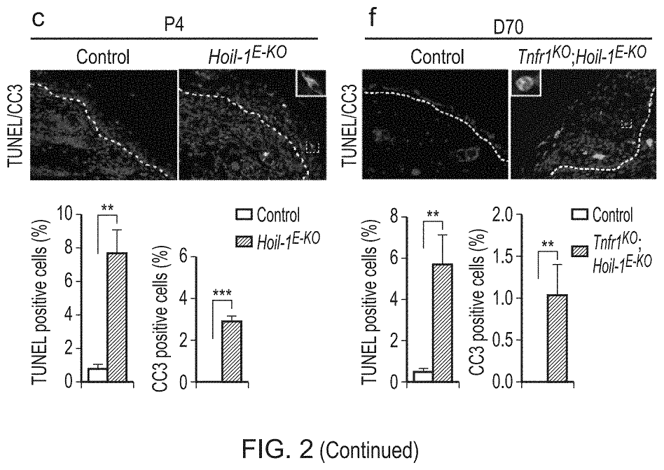

Extended Data FIG. 2: TNFR1 deficiency in Hoip^{E-KO} mice results in skin inflammation in adulthood. a, Kaplan-Meier survival curve of mice with the indicated genotypes. Comparisons between Hoip^{E-KO} (n=10) and Tnfr1^{KO};Hoip^{E-KO} (n=27) mice were submitted for statistical analysis. ***P≤0.001. Tnfr1^{KO};Hoip^{E-KO} mice were culled due to severe skin disease according to the regulations of the UK home office for animal welfare. b, Epidermal thickness quantification of skin sections from mice with the indicated genotypes at D70 (n=4 per genotype). Error bars represent mean values±s.e.m. ***P≤0.001. c, Flow cytometry analysis of immune cells in skin samples from mice with the indicated genotypes at D70. Bar graphs show the percentage of the indicated immune cell subpopulation relative to live and Side Scatter profile (n=5 per genotype). Error bars represent mean values±s.e.m. *P≤0.05, **P≤0.01, ***P≤0.001. Control mice represent a pool of Tnfr1^{KO};Hoip^{fl/fl};K14-Cre− and Tnfr1^{KO};Hoip^{fl/wt};K14-Cre+ mice (b, c).

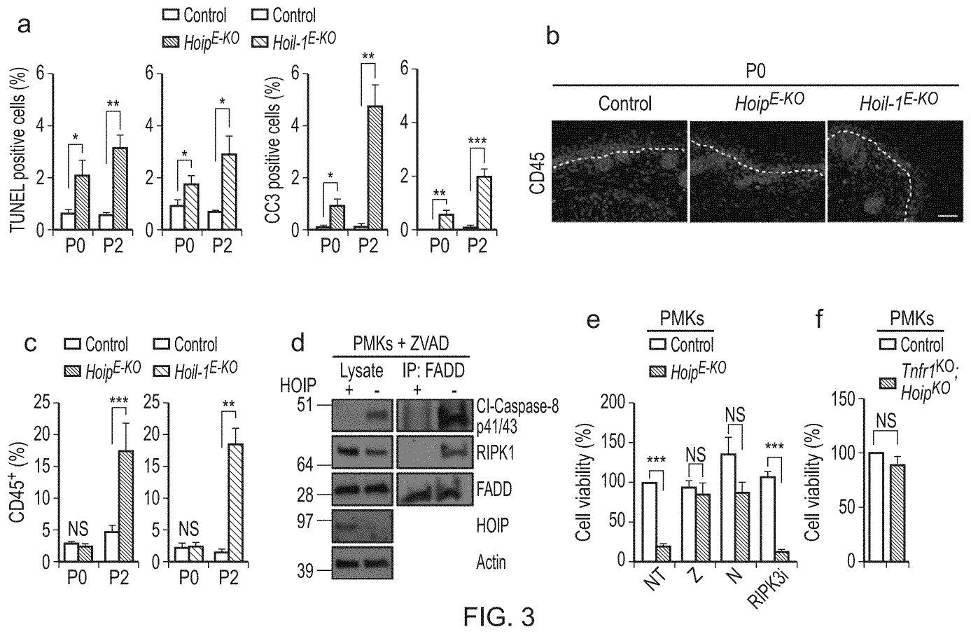

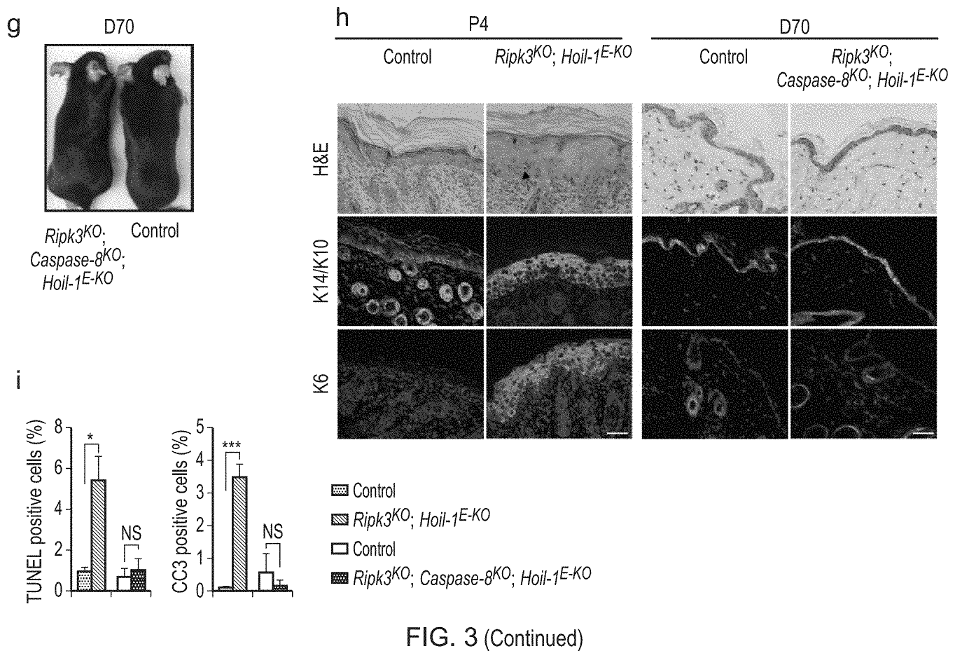

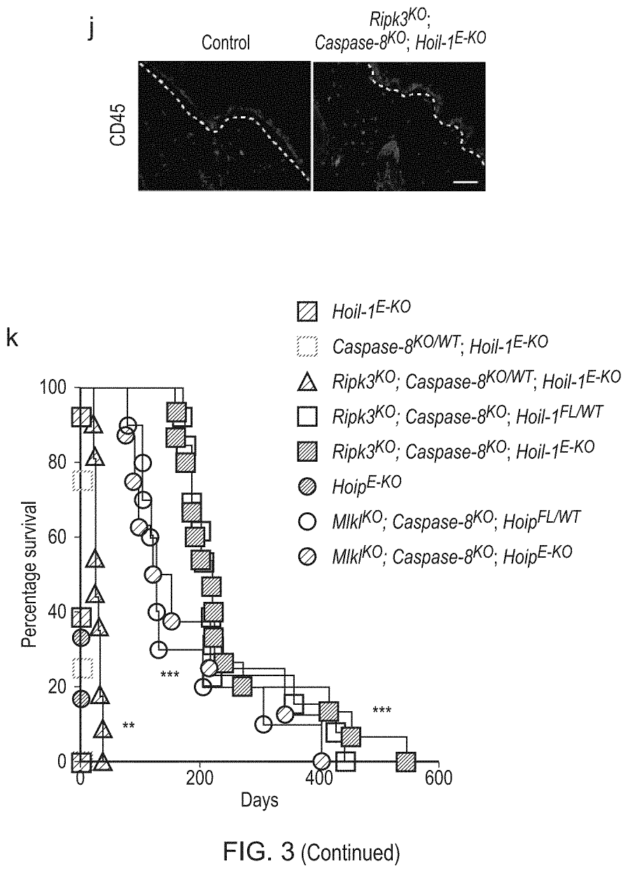

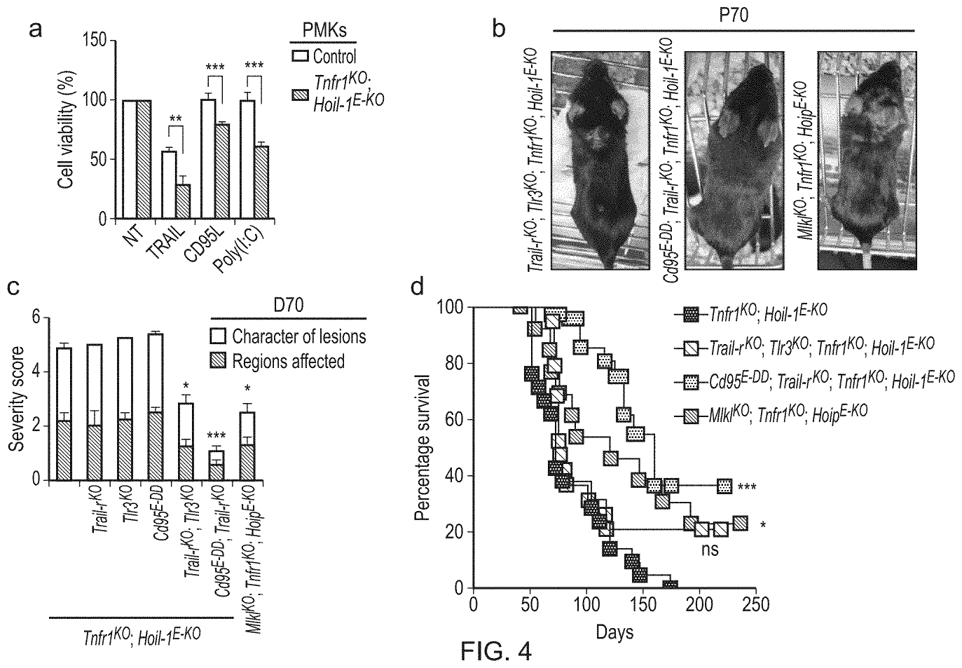

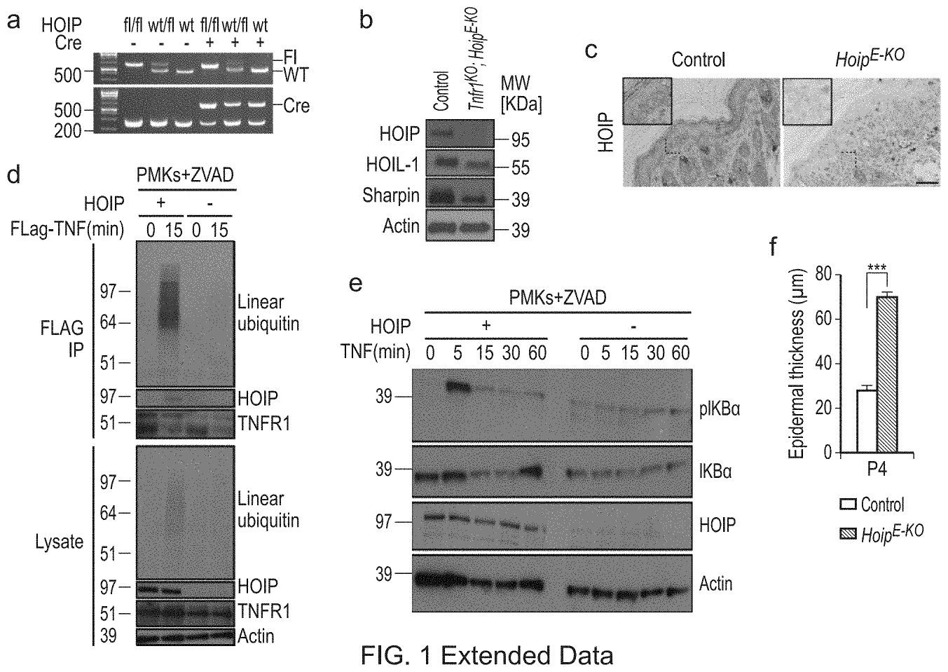

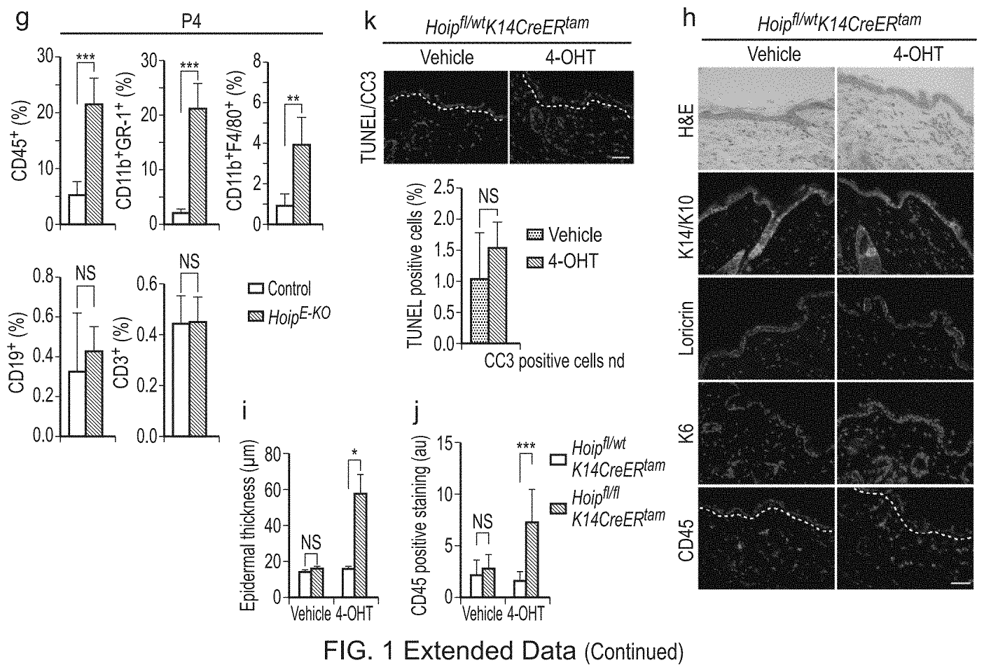

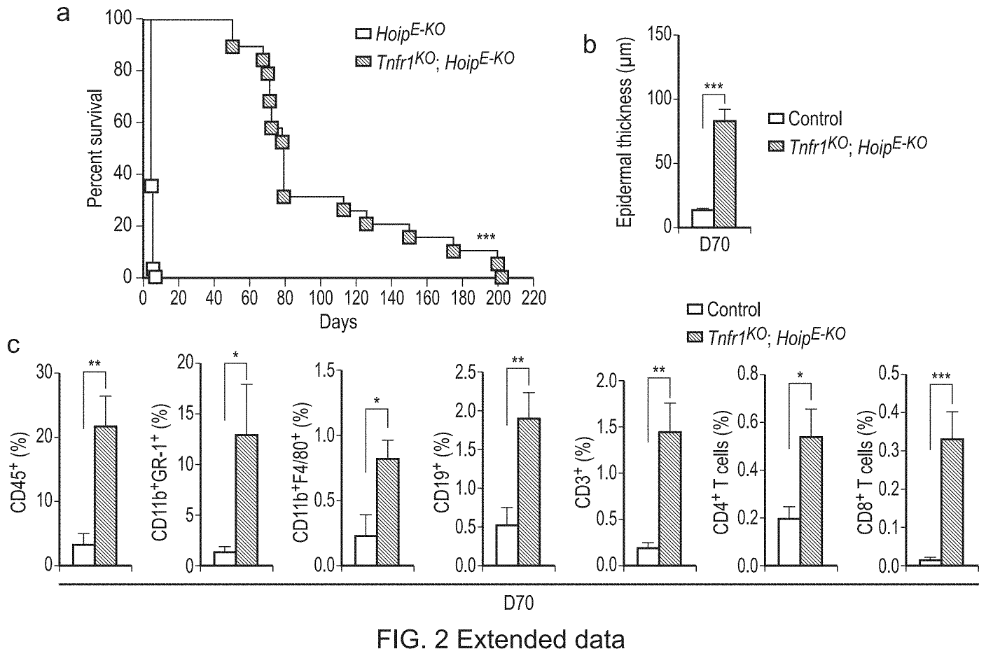

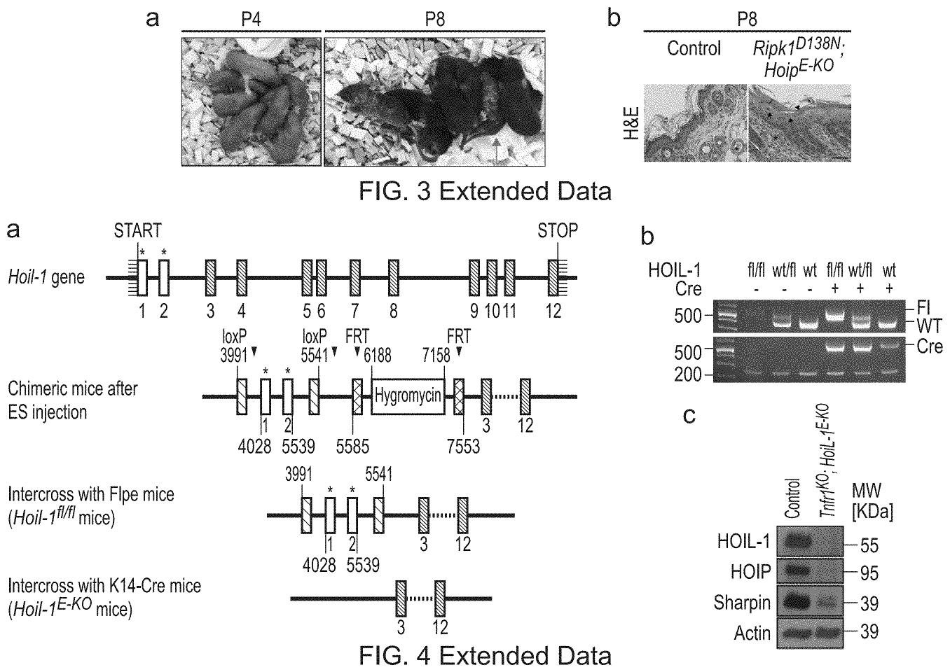

Extended Data FIG. 3: Genetic inhibition of the kinase activity of RIPK1 delays lethality of Hoip^{E-KO} mice by 4 days. a, Representative images of mice at the indicated postnatal days (n=8 mice per genotype). Arrows indicate RIPK1^{D138N};Hoip^{E-KO} mice at P8 (right panel). RIPK1^{D138N};Hoip^{E-KO} mice were indistinguishable from control littermates at P4 (left panel). b, Representative images of skin sections stained with H&E from mice with the indicated genotypes (n=3 mice per genotype). Arrows: pyknotic nuclei, stars: immune cell infiltrates, arrowhead: parakeratosis and black bar: hyperkeratosis. Scale bars, 50 μm. Control mice represent a pool of RIPK1^{D138N};Hoip^{fl/fl};K14-Cre− and RIPK1^{D138N};Hoip^{fl/wt};K14-Cre+ mice.

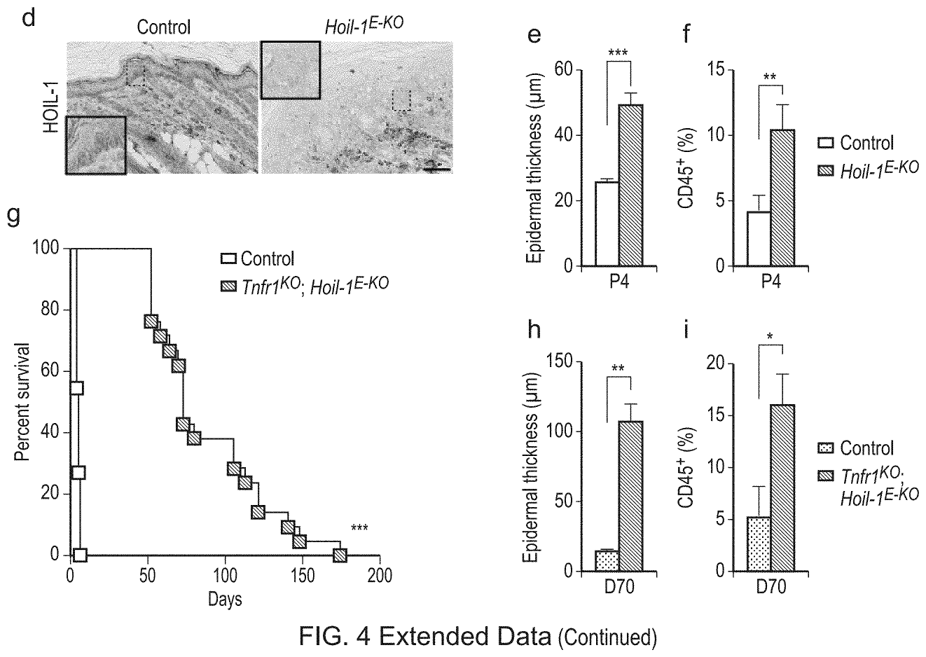

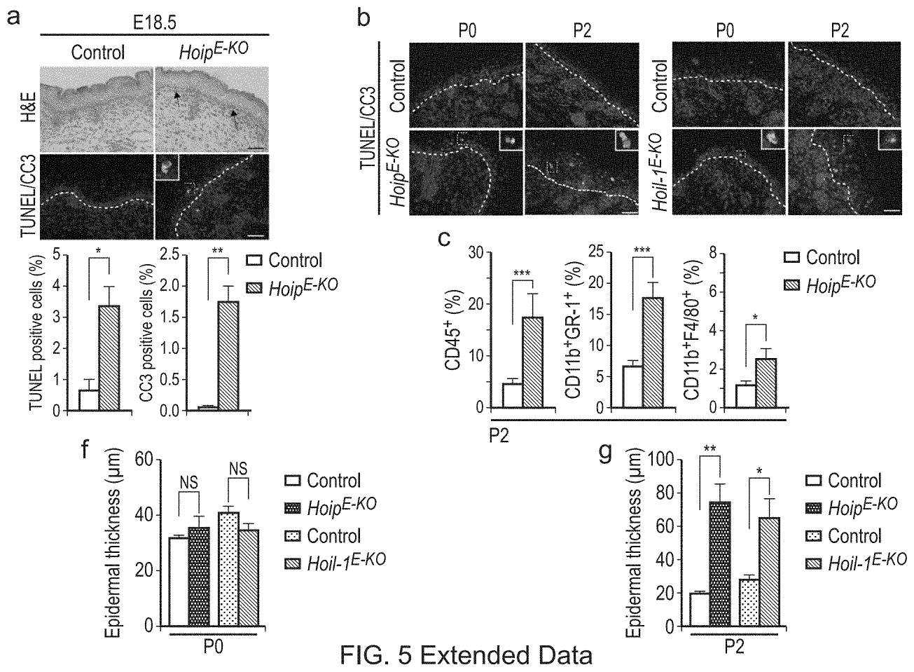

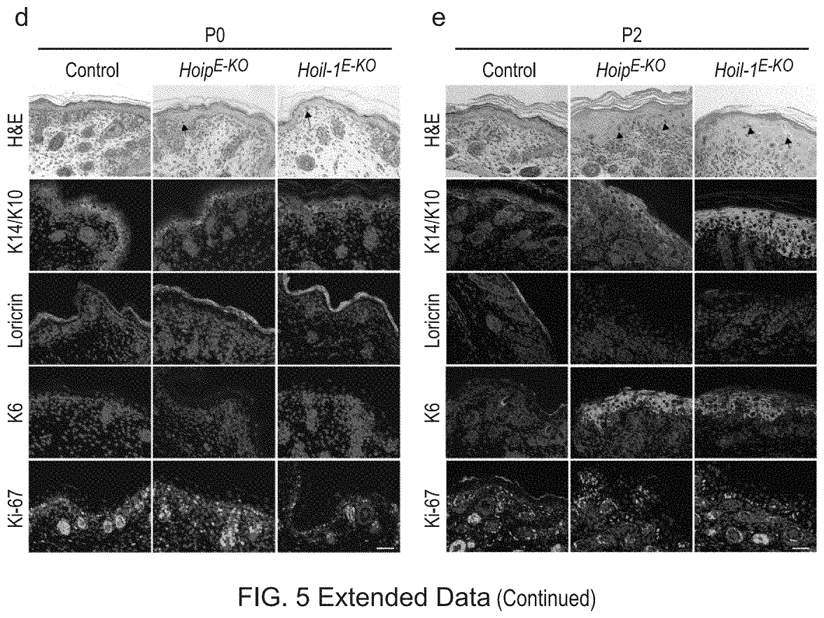

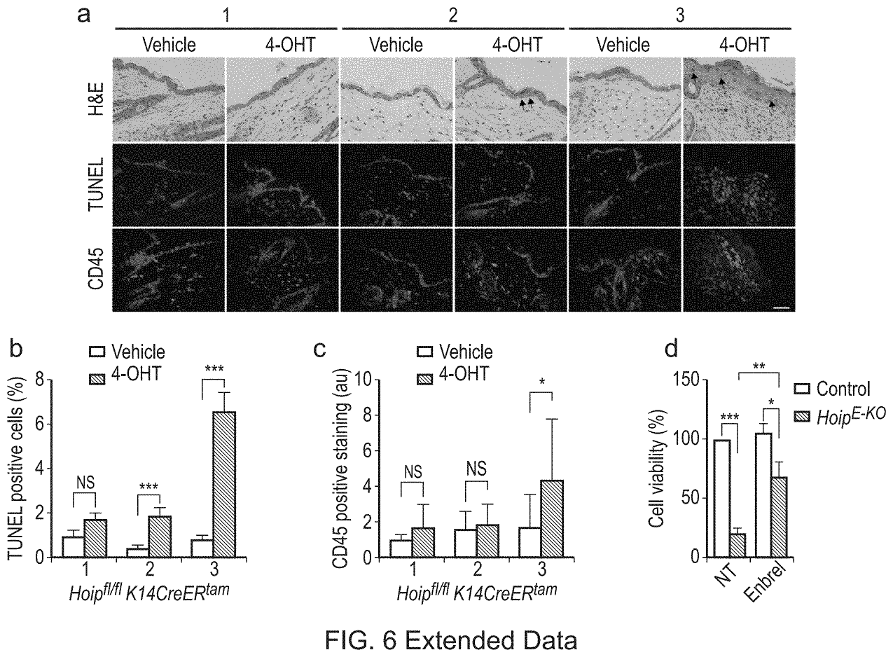

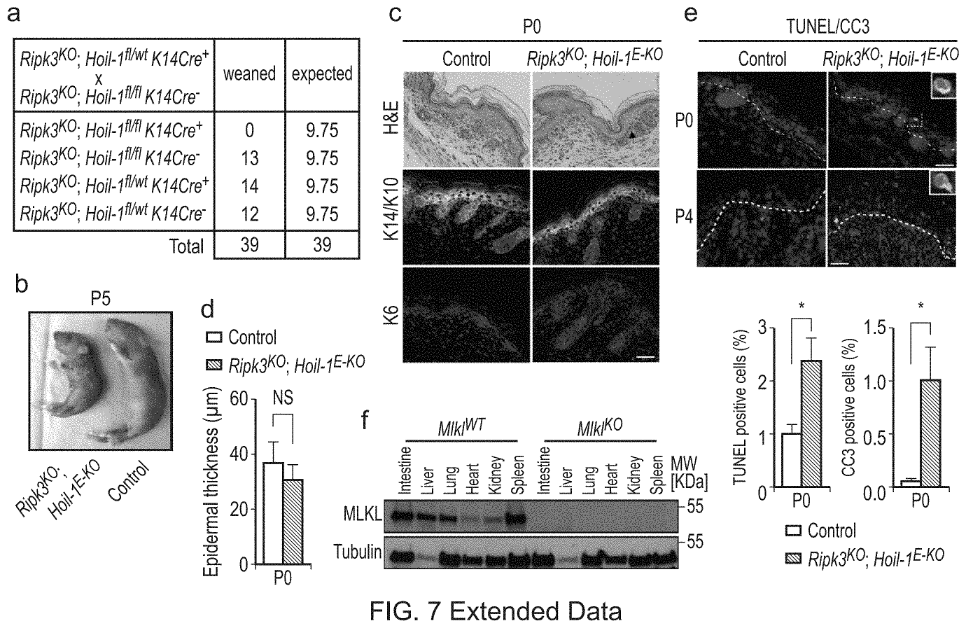

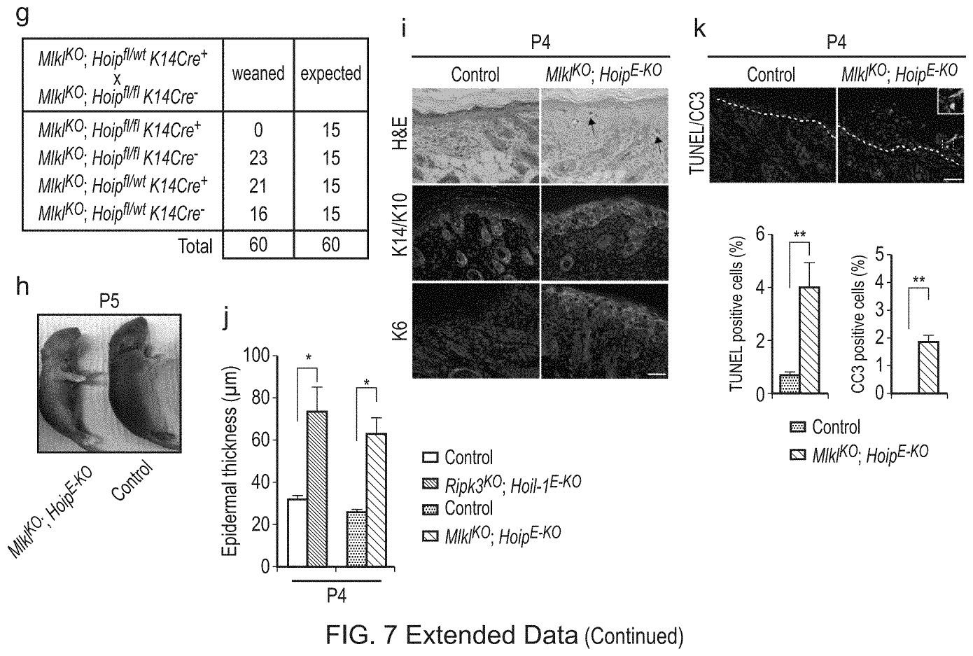

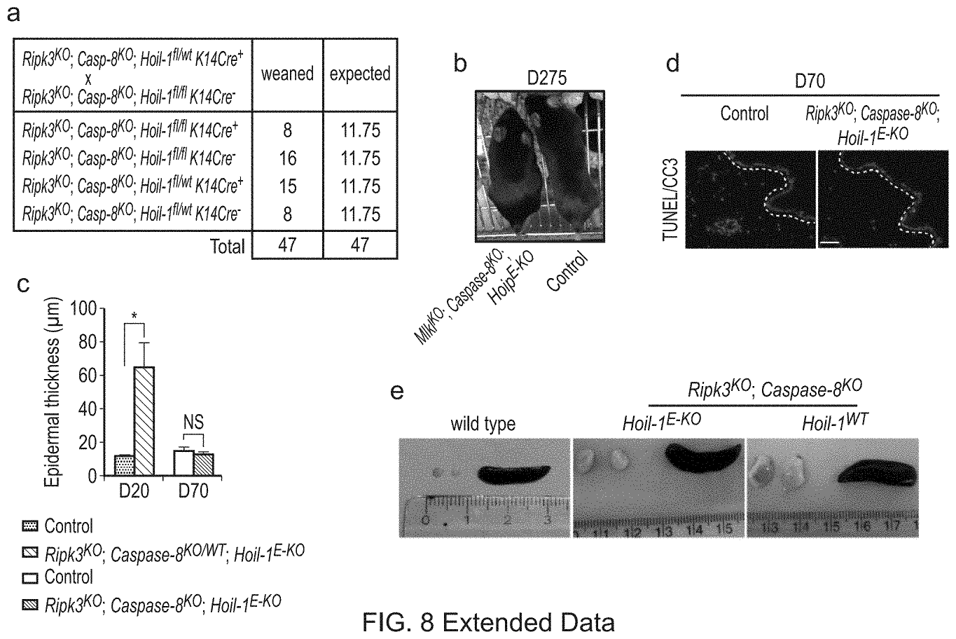

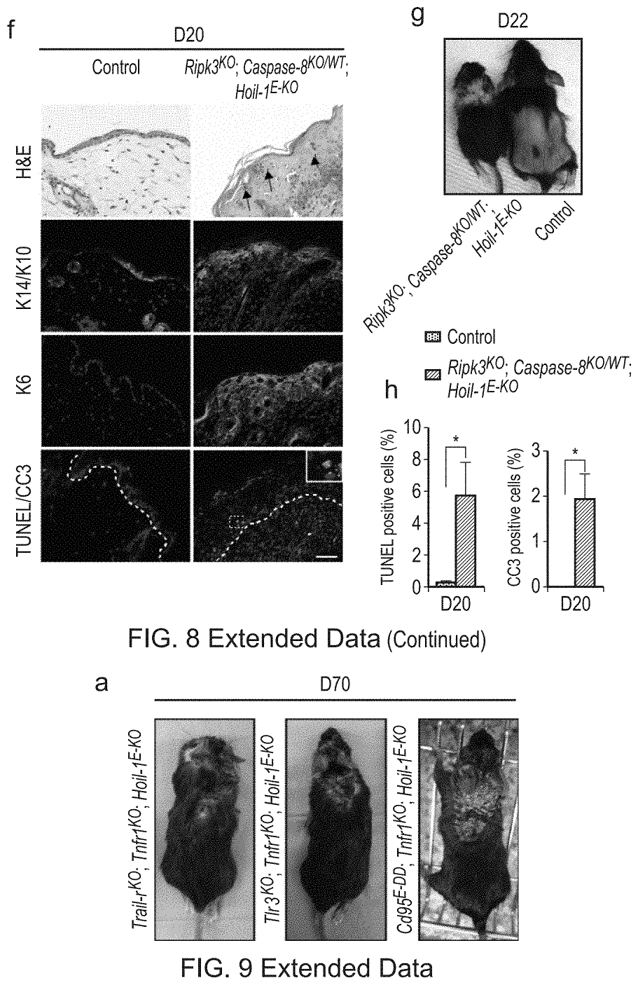

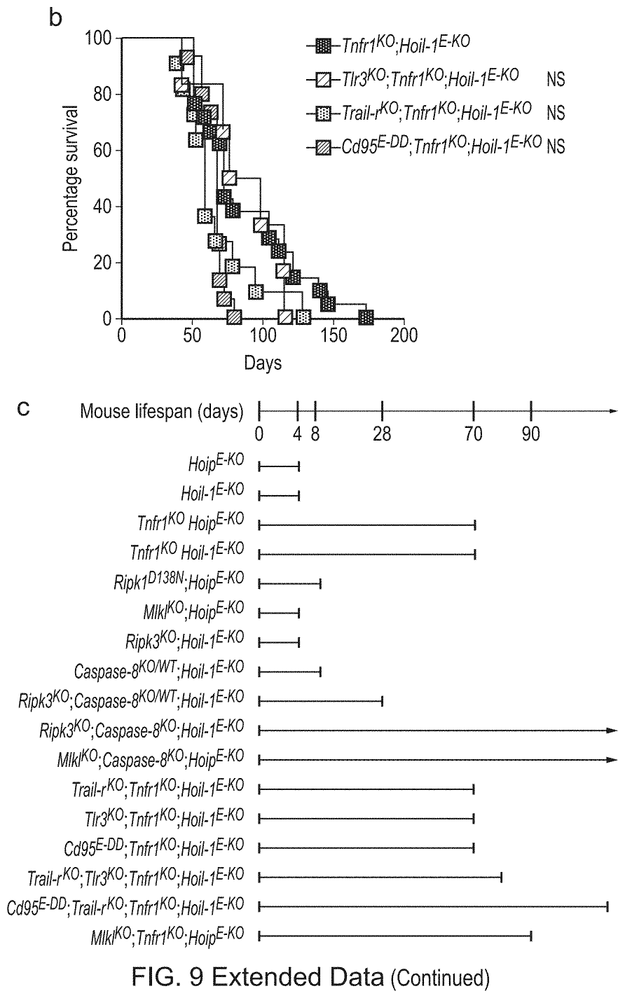

Extended Data FIG. 4: Generation and characterisation of Hoil-1^{E-KO} and Tnfr1^{KO};Hoil-1^{E-KO} mice. a, Schematic representation of the knockout strategy followed to generate Hoil-1^{E-KO} mice. b, PCR genotyping of DNA isolated from the ear punch of mice with the indicated genotypes. c, Western blot analysis of LUBAC components in PMKs derived from mice with the indicated genotypes. d, Representative images of skin sections stained with antibody against HOIL-1 at P4. Scale bar, 50 μm. e, h, Epidermal thickness quantification of skin sections from mice with the indicated genotypes at P4 (e) and D70 (h) (n=4 per genotype). Error bars represent mean values±s.e.m. **P≤0.01, ***P≤0.001. f, i, Flow cytometry analysis of immune cells in skin samples from mice with the indicated genotypes at P4 (f) and D70 (i). Bar graphs represent the percentage of CD45 positive cells relative to the Forward and Side scatter profile (n=5 per genotype). Error bars represent mean values±s.e.m. *P≤0.05, **P≤0.01. g, Kaplan-Meier survival curve of mice with the indicated genotypes. Comparisons between Hoil-1^{E-KO} (n=12) and Tnfr1^{KO};Hoil-1^{E-KO} (n=20) mice were submitted for statistical analysis. Tnfr1^{KO};Hoil-1^{E-KO} mice were culled according to the regulations of UK home office for animal welfare. ***P≤0.001. Control mice represent a pool of Hoil-1^{fl/fl};K14-Cre− and Hoil-1^{fl/wt};K14-Cre+ (d-f) or Tnfr1^{KO};Hoil-1^{fl/fl};K14-Cre− and Tnfr1^{KO};Hoil-1^{fl/wt};K14-Cre+ mice (c, g-i).

Extended Data FIG. 5: Analysis of Hoip^{E-KO} and Hoil-1^{E-KO} mice at different days. a, b Representative images of skin sections from Hoip^{E-KO} mice with the indicated stainings and corresponding quantification, TUNEL (red) and CC3 (green) at the indicated times. Nuclei were stained with DAPI (blue). White dashed lines separate the epidermis (above) from the dermis (below). Arrows indicate pyknotic nuclei. Scale bars, 50 μm. Error bars represent mean values±s.e.m. *P≤0.05, **P≤0.01 (n=3 mice per genotype). c, Flow cytometric analysis of immune cells in skin samples from mice with the indicated genotypes at P2. Bar graphs represent the percentage of the indicated immune cell subpopulation relative to the Forward and Side scatter profile (n=5 per genotype). Error bars represent mean values±s.e.m. *P≤0.05, ***P≤0.001. d, e, Representative images of skin sections of mice with the indicated genotypes stained as indicated at P0 (d) and P2 (f) (n=3 mice per genotype). Arrows indicate pyknotic nuclei. Nuclei were stained with DAPI (blue). Scale bars, 50 μm. f, g, Epidermal thickness quantification of skin sections from mice with the indicated genotypes at P0 (f) and P2 (g) (n=3 per genotype). Error bars represent mean values±s.e.m. *P≤0.05, **P≤0.01, NS=not significant. CC3: cleaved Caspase-3. Control mice represent a pool of Hoip^{fl/fl};K14-Cre− and Hoip^{fl/wt};K14-Cre+ or Hoil-1^{fl/fl};K14-Cre− and Hoil-1^{fl/wt};K14-Cre+ (a-g).

Extended Data FIG. 6: Cell death precedes inflammation when HOIP is deleted in keratinocytes of adult mice. a, Representative images of Hoip^{fl/fl}K14CreER^{tam} mice analysed after one, two or three treatments with vehicle or 4-OHT and stained as indicated (n=3 per genotype). Arrows: pyknotic nuclei, star: immune infiltrates. Nuclei were stained with DAPI (blue). Scale bar, 50 μm. b, Quantification of TUNEL positive cells in skin sections of Hoip^{fl/fl}K14CreER^{tam} mice treated as in (a) (n=3 per genotype). Error bars represent mean values±s.e.m. ***P≤0.001, NS=not significant. c, Quantification of CD45 staining in skin sections from Hoip^{fl/fl}K14CreER^{tam} mice treated as in a was performed by measuring overall fluorescence intensity using ImageJ. NS=not significant. (n=3 mice per genotype). au=arbitrary units. d, PMKs derived from Hoip^{E-KO} and control mice were cultured with or without (NT) Etanercept (Enbrel®) [50 μg/ml]. Cell viability (%) was measured by CellTiter-Glo assay. Results are expressed as mean values±s.e.m. (n=7 mice per genotype). 0.05, **P≤0.01, ***P≤0.001. Control mice represent a pool of Hoip^{fl/fl};K14-Cre− and Hoip^{fl/wt};K14-Cre+.

Extended Data FIG. 7: Loss of RIPK3/MLKL-mediated necroptosis does not affect the phenotype of LUBAC-specific-keratinocyte-deficient mice. a, g, Table depicting genotype statistics of animals obtained after the crossing of mice with the indicated genotypes. Numbers of animals obtained (weaned) and expected, according to the Mendelian frequencies, are reported. b, h, Representative images of mice with the indicated genotypes at P5. c, i, Representative images of skin sections of mice with the indicated genotypes stained as indicated at P0 (c) and at P4 (i) (n=4 per genotype). Arrows indicate pyknotic nuclei. Nuclei were stained with DAPI (blue). Scale bar, 50 μm. d, j, Epidermal thickness quantification of skin sections from mice with the indicated genotypes at P0 (d) and at P4 (j) (n=4 per genotype). Error bars represent mean values±s.e.m. *P≤0.05, NS=not significant. e, k, Representative images of skin sections double stained with TUNEL (red) and CC3 antibody (green) in mice with the indicated genotypes (top panels). Nuclei were stained with DAPI (blue). White dashed lines separate the epidermis (above) from the dermis (below). Scale bars, 50 μm. Quantification of TUNEL and CC3 positive cells in skin sections from mice with the indicated genotypes (bottom panels) (n=3 per genotype). Error bars represent mean values±s.e.m. *P≤0.05, **P≤0.01. CC3: cleaved Caspase-3. f, Western blot analysis of MLKL expression in the indicated organs derived from mice with the indicated genotypes. Control mice represent a pool of Ripk3^{KO};Hoil-1^{fl/fl};K14-Cre− and Ripk3^{KO};Hoil-1^{fl/wt};K14-Cre+ (b-e) and Mlkl^{KO};Hoip^{fl/fl};K14-Cre− and Mlkl^{KO};Hoip^{fl/wt};K14-Cre+ mice (h-k).

Extended Data FIG. 8: Combined deletion of RIPK3 and Caspase-8 fully prevents the lethal inflammatory phenotype of Hoil-1^{E-KO} mice. a, Table depicting genotype statistics of animals obtained after the crossing of mice with the indicated genotypes. Numbers of animals obtained (weaned) and expected, according to the Mendelian frequencies, are reported. b, g, Representative images of mice with the indicated genotypes (n=4 (b) and 11 (g) per genotype). c, Epidermal thickness quantification of skin sections from mice with the indicated genotypes at the specified days after birth (n=3 per genotype). Error bars represent mean values±s.e.m. *P≤0.05, NS=not significant. d, Representative images of skin sections double stained with TUNEL (red) and CC3 antibody (green) in mice with the indicated genotypes. Nuclei were stained with DAPI (blue). White dashed lines separate the epidermis (above) from the dermis (below). Scale bars, 50 μm. e, Representative images of axial lymph nodes and spleen from mice with the indicated genotypes at around 7 months. f, Representative images of skin sections of mice with the indicated genotypes stained as indicated at D20 (n=3 per genotype). Arrows indicate pyknotic nuclei. Nuclei were stained with DAPI (blue). Scale bar, 50 μm. h, Quantification of TUNEL and CC3 positive cells in skin sections from mice with the indicated genotypes (n=3 per genotype). Error bars represent mean values±s.e.m. *P≤0.05. CC3: cleaved Caspase-3. Control mice represent a pool of Mlkl^{KO};Caspase-8^{KO};Hoip^{fl/fl};K14-Cre− and Mlkl^{KO};Caspase-8^{KO};Hoip^{fl/wt};K14-Cre+ (b) or Ripk3^{KO};Caspase-8^{KO}; Hoil-1^{fl/fl};K14-Cre−, Ripk3^{KO};Caspase-8^{KO};Hoil-1^{fl/wt};K14-Cre+ or Ripk3^{KO};Caspase-8^{KO/WT};Hoil-1^{fl/fl};K14-Cre− and Ripk3^{KO};Caspase-8^{KO/WT};Hoil-1^{fl/wt};K14-Cre+ mice (c, d, f-h).

Extended Data FIG. 9: TLR3, DD of CD95 or TRAIL-R deletion alone is not sufficient to prevent TNFR1-independent dermatitis. a, Representative images of mice with the indicated genotypes. b, Kaplan-Meier survival curve, comparison between Tnfr1^{KO};Hoil-1^{E-KO} mice and mice with the indicated genotypes were submitted to statistical analysis. Tnfr1^{KO};Hoil-1^{E-KO} (n=21), Trail-r^{KO};Tnfr1^{KO};Hoil-1^{E-KO} (n=11), Tlr3^{KO}Tnfr1^{KO};Hoil-1^{E-KO} (n=6) and Cd95^{E-DD};Tnfr1^{KO};Hoil-1^{E-KO} (n=15). c, Lifespan of mice with indicated genotypes.

### EXAMPLES

### Example 1—Summary

We have now developed disease models in mice in which the animals develop a more severe form of inflammatory skin disease than in the model we employed in our studies in 2011 and 2013^{6,7}.

Specifically, SHARPIN, a component of the linear ubiquitin chain assembly complex (LUBAC)⁶⁻⁹, prevents inflammation by inhibiting TNF-induced RIPK1 kinase activity-dependent cell death^{7,8,10}.

In the present models, we show that keratinocyte-specific loss in either of the other two LUBAC components, HOIP or HOIL-1¹¹⁻¹³ (Hoip^{E-KO} and Hoil-1^{E-KO} mice), results in postnatal lethal skin inflammation.

In contrast to the SHARPIN-mutant animals, in Hoip^{E-KO} and Hoil-1^{E-KO} mice, loss of TNFR1 did not abrogate, but merely delayed, lethal dermatitis. Genetic ablation of TNFR1 completely inactivates cell death induction, but also gene activation, via this receptor. This means that in these new models TNFR1-mediated signalling contributes to the inflammation but is not solely responsible for it.

We found that combined constitutive loss of TNFR1 with either constitutive loss of TRAIL-R, TLR3 or with specific loss of the death domain (DD) of CD95 in keratinocytes did not result in any further delay in onset of inflammation as compared to when TNFR1 was constitutively deleted.

Strikingly, however, the constitutive deletion of TNFR1, when combined with constitutive deletion of TRAIL-R and specific deletion of the DD of CD95 in keratinocytes unexpectedly prevented the development of any inflammatory syndrome in the resulting mice.

Thus, in the absence of TNFR1, CD95L and TRAIL are together responsible for causing lethal dermatitis by inducing cell death.

Moreover, we also discovered that combined loss of TNFR1 with that of TRAIL-R and TLR3 significantly ameliorates the severe skin inflammatory disease even though it does not completely prevent skin inflammation

Collectively, this study unveils aberrant death receptor-mediated cell death as the aetiology of dermatitis and sheds new light on the mechanisms of auto-inflammation and auto-immunity that occur in the absence of TNFR1 or when TNF is blocked.

Our results further suggest that autoimmune patients whose disease has a cell death aetiology but is (currently thought to be) refractory to TNF inhibition, may benefit from combining TNF inhibition with that of TRAIL and CD95L, or other targets as described herein. Importantly, this new treatment paradigm may extend beyond auto-immune diseases in which TNF inhibition is currently used successfully.

### REFERENCES FOR DESCRIPTION AND EXAMPLE 1

1 Kalliolias, G. D. & Ivashkiv, L. B. TNF biology, pathogenic mechanisms and emerging therapeutic strategies. Nature reviews. Rheumatology 12, 49-62, doi:10.1038/nrrheum.2015.169 (2016).
2 Monaco, C., Nanchahal, J., Taylor, P. & Feldmann, M. Anti-TNF therapy: past, present and future. International immunology 27, 55-62, doi:10.1093/intimm/dxu102 (2015).
3 Lopetuso, L. R. et al. Can We Predict the Efficacy of Anti-TNF-alpha Agents? Int J Mol Sci 18, doi:10.3390/ijms18091973 (2017).
4 Cho, J. H. & Feldman, M. Heterogeneity of autoimmune diseases: pathophysiologic insights from genetics and implications for new therapies. Nature medicine 21, 730-738, doi:10.1038/nm.3897 (2015).
5 Roda, G., Jharap, B., Neeraj, N. & Colombel, J. F. Loss of Response to Anti-TNFs: Definition, Epidemiology, and Management. Clin Transl Gastroenterol 7, e135, doi:10.1038/ctg.2015.63 (2016).
6 Gerlach, B. et al. Linear ubiquitination prevents inflammation and regulates immune signalling. Nature 471, 591-596, doi:10.1038/nature09816 (2011).
7 Rickard, J. A. et al. TNFR1-dependent cell death drives inflammation in Sharpin-deficient mice. eLife 3, doi:10.7554/eLife.03464 (2014).
8 Walczak, H. TNF and ubiquitin at the crossroads of gene activation, cell death, inflammation, and cancer. Immunological reviews 244, 9-28, doi:10.1111/j.1600-065X.2011.01066.x (2011).
9 Peltzer, N., Darding, M. & Walczak, H. Holding RIPK1 on the Ubiquitin Leash in TNFR1 Signaling. Trends in cell biology, doi:10.1016/j.tcb.2016.01.006 (2016).
10 Ward-Kavanagh, L. K., Lin, W. W., Sedy, J. R. & Ware, C. F. The TNF Receptor Superfamily in Co-stimulating and Co-inhibitory Responses. Immunity 44, 1005-1019, doi:10.1016/j.immuni.2016.04.019 (2016).
11 Zinngrebe, J. & Walczak, H. TLRs Go Linear—On the Ubiquitin Edge. Trends in molecular medicine 23, 296-309, doi:10.1016/j.molmed.2017.02.003 (2017).
12 Vanden Berghe, T., Linkermann, A., Jouan-Lanhouet, S., Walczak, H. & Vandenabeele, P. Regulated necrosis: the expanding network of non-apoptotic cell death pathways. Nature reviews. Molecular cell biology 15, 135-147, doi:10.1038/nrm3737 (2014).
13 von Karstedt, S., Montinaro, A. & Walczak, H. Exploring the TRAILs less travelled: TRAIL in cancer biology and therapy. Nature reviews. Cancer 17, 352-366, doi:10.1038/nrc.2017.28 (2017).
14 Wick, W. et al. A phase II, randomized, study of weekly APG101+reirradiation versus reirradiation in progressive glioblastoma. Clinical cancer research: an official journal of the American Association for Cancer Research 20, 6304-6313, doi:10.1158/1078-0432.CCR-14-0951-T (2014).
15 Tuettenberg, J. et al. Pharmacokinetics, pharmacodynamics, safety and tolerability of APG101, a CD95-Fc fusion protein, in healthy volunteers and two glioma patients. Int Immunopharmacol 13, 93-100, doi:10.1016/j.intimp.2012.03.004 (2012).
17 Cheng, K., Wang, X. & Yin, H. Small-molecule inhibitors of the TLR3/dsRNA complex. J Am Chem Soc 133, 3764-3767, doi:10.1021/ja111312h (2011).

### Example 2—Mammalian Models of Inflammation

LUBAC is a key regulator of gene activation and cell death pathways triggered by several innate and adaptive immune receptors, including TNFR1¹⁹⁻²¹. Mice deficient for SHARPIN, referred to as chronic proliferative dermatitis mice (cpdm), suffer from severe skin inflammation²²⁻²⁴ that is caused by aberrant TNF/TNFR1-induced RIPK1 kinase activity-dependent cell death^{7,8,10,25}.

HOIP is the central and catalytically active LUBAC component^{11,13} and its deficiency results in embryonic lethality²⁶. To understand the role of HOIP in skin homeostasis we generated mice that lack HOIP selectively in epidermal keratinocytes (Hoip^{E-KO} mice) (Extended Data FIG. 1a-c). HOIP deficiency abrogated linear ubiquitination at the TNFR1 signalling complex (TNFR1-SC) (Extended Data FIG. 1d) and diminished TNFR1-mediated NF-κB activation in primary murine keratinocytes (PMKs) from Hoip^{E-KO} mice (Extended Data FIG. 1e). These mice rapidly developed severely damaged and scaly skin, which invariably resulted in their death between P4 and P6 (FIG. 1a). Histological analysis of Hoip^{E-KO} mice at P4 revealed increased epidermal thickness, parakeratosis, hyperkeratosis and keratinocyte differentiation defects (FIG. 1b and Extended Data FIG. 10. These features were accompanied by myeloid cell infiltration and high levels of cell death as demonstrated by increased cleaved caspase-3 and TUNEL staining (FIG. 1b, c and Extended Data FIG. 1g). Together, these observations reveal that Hoip^{E-KO} mice develop a fatal dermatitis characterised by inflammation and aberrant keratinocyte death.

To assess the impact of acute deletion of HOIP in keratinocytes, we treated adult Hoip^{fl/fl}K14CreER^{Tam} adult mice with 4-Hydroxytamoxifen (4-OHT) in a localised area of the skin. These skin areas showed epidermal thickening, hyperplasia, hyper- as well as parakeratosis and keratinocyte differentiation defects (FIG. 1d, e and Extended Data FIG. 1h, i), accompanied by increased immune cell infiltration and cell death (FIG. 1e, f and Extended Data FIG. 1h, j, k). This is reminiscent of the skin phenotype of Hoip^{E-KO} mice, demonstrating that HOIP is also required to maintain skin homeostasis in adult mice.

### Example 3—TNFR1, and RIPK1 in Mammalian Model of Inflammation

Since the inflammatory phenotype observed in cpdm mice is completely rescued by the absence of TNF, TNFR1 or by a kinase dead version of RIPK1^{7,8,10} we next tested whether genetic ablation of TNFR1 or of the kinase activity of RIPK1 could also prevent the morbidity and mortality in Hoip^{E-KO} mice.

Unexpectedly, however, inflammation was only delayed in Tnfr1^{KO};Hoip^{E-KO} mice as they progressively developed severe skin lesions resulting in a median survival of 70 days (FIG. 1g and Extended Data FIG. 2a). Sick Tnfr1^{KO};Hoip^{E-KO} mice presented with epidermal disruption, thickening, parakeratosis and hyperkeratosis (FIG. 1h and Extended Data FIG. 2b). Crucially, infiltration by myeloid and lymphoid cells and cell death were significantly augmented in the epidermis of adult Tnfr1^{KO};Hoip^{E-KO} mice compared to control animals (FIG. 1h, i and Extended Data FIG. 2c).

Surprisingly, genetic ablation of the kinase activity of RIPK1 was even less efficient than TNFR1 ablation in preventing fatal dermatitis as RIPK1^{D138N};Hoip^{E-KO} mice died at around P8 showing signs of severe skin disease (Extended Data FIG. 3). Thus, lethal dermatitis caused by HOIP deficiency in keratinocytes is mediated only in part by the kinase activity of RIPK1 and occurs even in the absence of TNFR1.

### Example 4—Further Mammalian Model of Inflammation

We next examined the role of HOIL-1, the third LUBAC component, in skin homeostasis. Although HOIL-1-deficient mice generated elsewhere were reported to be healthy²⁷, we found that absence of HOIL-1 in keratinocytes (Hoil-1^{E-KO} mice) (Extended Data FIG. 4a-d) resulted in postnatal lethality caused by severe dermatitis with increased epidermal cell death (FIG. 2a-c and Extended Data FIG. 4e, f). This recapitulated the phenotype of Hoip^{E-KO} mice, demonstrating that HOIL-1 is as important as HOIP in preventing epidermal cell death and lethal skin inflammation. This finding is consistent with our recent observation that, like constitutive loss of HOIP²⁶, also that of HOIL-1 causes embryonic lethality (Peltzer et al., manuscript in revision).

In line with the finding of Example 3, adult Tnfr1^{KO};Hoil-1^{E-KO} mice showed a median survival of 70 days after developing dermatitis characterised by increased immune cell infiltration and epidermal cell death resembling the phenotype of Tnfr1^{KO};Hoip^{E-KO} mice (FIG. 2d-f and Extended Data FIG. 4g-i). This demonstrates that in the case of keratinocyte-specific deletion of either HOIP or HOIL-1, the impact on skin inflammation extends beyond the regulation of TNFR1 signalling.

We next investigated the temporal relationship between aberrant cell death and inflammation in Hoip^{E-KO} and Hoil-1^{E-KO} mice. Increased cell death in the epidermis of Hoip^{E-KO} and Hoil-1^{E-KO} mice was already apparent in utero at E18.5 and at birth (P0) (FIG. 3a and Extended Data FIG. 5a, b). This implies that lack of linear ubiquitination in keratinocytes results in aberrant cell death in sterile conditions. Hoip^{E-KO} and Hoil-1^{E-KO} mice displayed abnormally increased immune cell infiltration only at P2 and P4 but not at birth (FIG. 1, 2, 3b, c and Extended Data FIG. 1g, 4f, 5c). Accordingly, keratinocyte differentiation and epidermal thickness appeared aberrant at P2 and P4, but not at E18.5 or P0 (FIG. 1, 2 and Extended Data FIG. 5a, d-g).

Moreover, 4-OHT treated Hoip^{fl/fl}K14CreER^{Tam} mice consistently exhibited increased cell death before immune cell infiltration became apparent (Extended Data FIG. 6a-c). Thus, excessive cell death precedes an inflammatory response, suggesting that cell death triggers lethal dermatitis upon loss of HOIP or HOIL-1 in keratinocytes.

### Example 5—Mechanism of Cell Death Induction in Mammalian Model of Inflammation

To understand the mechanism of cell death induction in the skin of Hoip^{E-KO} and Hoil-1^{E-KO} mice, we first analysed the formation of the signalling platforms known to trigger cell death downstream of death receptors²⁸ by immunoprecipitating the adaptor protein FADD in PMKs derived from these animals. This revealed that, even without an exogenous stimulus, a FADD/Caspase-8/RIPK1-containing complex was readily detectable in HOIP-deficient but not in control PMKs (FIG. 3d). Consistent with apoptotic signalling by such a complex, the HOIP-deficient cells were also less viable in the absence of exogenous stimuli (FIG. 3e).

This loss in viability was prevented by inhibition of caspases or RIPK1 activity by incubation with ZVAD or necrostatin-1s respectively, but not inhibition of RIPK3 activity (FIG. 3e).

Genetic ablation of TNFR1 or the inhibition of TNF also restored viability (FIG. 3f and Extended Data FIG. 6d). These results indicate that in PMKs HOIP prevents aberrant RIPK1 kinase-dependent apoptosis triggered by autocrine TNF. Yet, in vivo the regulation of apoptosis seems to be more complex since genetic ablation of RIPK1 kinase activity or TNFR1 did not prevent dermatitis of Hoip^{E-KO} mice.

### Example 6—Role of Apoptosis and Necroptosis in Cell Death Induction in Mammalian Model of Inflammation

To evaluate whether excessive cell death could be causative for the lethal dermatitis in Hoip^{E-KO} and Hoil-1^{E-KO} mice, we first explored the role of necroptosis.

Consistent with the apoptotic cell death observed in vitro, genetic ablation of Ripk3 in Hoil-1^{E-KO} and that of Mlkl in Hoip^{E-KO} mice failed to prevent cell death and skin inflammation that leads to postnatal lethality (FIG. 3h, i and Extended Data FIG. 7).

We therefore next addressed the role of apoptosis by deleting Caspase-8 in Mlkl^{KO};Hoip^{E-KO} and Ripk3^{KO};Hoil-1^{E-KO} mice. Remarkably, both Mlkl^{KO};Caspase-8^{KO}; Hoip^{E-KO} and Ripk3^{KO};Caspase-8^{KO};Hoil-1^{E-KO} mice reached adulthood without any signs of skin disease (FIG. 3g and Extended Data FIG. 8a-b). Consistently, epidermal structure and keratinocyte differentiation were completely normal in Ripk3^{KO};Caspase-8^{KO}; Hoil-1^{E-KO} mice and these animals neither exhibited increased cell death nor immune cell infiltration in their skin (FIG. 3h-j and Extended Data FIG. 8c,d). Ripk3^{KO};Caspase-8^{KO}; Hoil-1^{E-KO} mice survived well beyond the 70 days when Tnfr1^{KO};Hoil-1^{E-KO} mice succumbed to severe dermatitis (FIG. 3k and Extended Data FIG. 4g) but had to be sacrificed because of lymphadenopathy and splenomegaly (Extended Data FIG. 8e), as previously reported for mice deficient in RIPK3 and Caspase-8^{29,30}.

Of note, heterozygosity of Caspase-8 was able to extend the survival of Hoil-1^{E-KO} mice to P7-P9 (FIG. 3k) and Ripk3^{KO};Caspase-8^{KO/WT};Hoil-1^{E-KO} mice developed fatal dermatitis around day 20 (FIG. 3k and Extended Data FIG. 8f-h). Collectively, these results demonstrate that Caspase-8-mediated apoptosis is causative for the lethal dermatitis in mice lacking HOIP or HOIL-1 in keratinocytes. By contrast, necroptosis only contributes to skin inflammation in Caspase-8^{KO/WT}; Hoil-1^{E-KO} mice, without being solely responsible for it as a substantial apoptotic component remains.

### Example 7—TNFR1-Independent Cell Death in Mammalian Model of Inflammation

We then studied the TNFR1-independent cell death causative for the fatal inflammation in LUBAC-keratinocyte-specific-deficient mice.

We first aimed to identify the mediators of cell death in Tnfr1^{KO};Hoil-1^{E-KO} mice.

PMKs derived from Tnfr1^{KO};Hoil-1^{E-KO} mice showed decreased cell viability as compared to control upon TNF-related apoptosis-inducing ligand (TRAIL), CD95 (Fas/APO-1) ligand (CD95L) or Polyinosinic:polycytidylic acid (Poly(I:C)) stimulation (FIG. 4a), consistent with our previous findings in other cell types^{21,32}. We, therefore, next genetically ablated TRAIL-R or TLR3 systemically or the death domain (DD) of CD95 specifically in keratinocytes in Tnfr1^{KO};Hoil-1^{E-KO} mice. Unfortunately, however, the resulting Trail-r^{KO};Tnfr1^{KO};Hoil-1^{E-KO}, Tlr3^{KO};Tnfr1^{KO};Hoil-1^{E-KO} and Cd95^{E-DD};Tnfr1^{KO};Hoil-1^{E-KO} mice all suffered from skin lesions which were indistinguishable in intensity from those of Tnfr1^{KO};Hoil-1^{E-KO} mice (FIG. 4c and Extended Data FIG. 9a, b).

Despite this discouraging result, we co-deleted TRAIL-R and TLR3 in Tnfr1^{KO};Hoil-1^{E-KO} mice and observed a significant, albeit transient, amelioration of skin inflammation in the resulting Trail-r^{KO};Tlr3^{KO};Tnfr1^{KO};Hoil-1^{E-KO} mice at D70 (FIG. 4b,c). However, these mice succumbed to inflammatory skin disease at around D80 (FIG. 4d).

We next combined loss of TRAIL-R with keratinocyte-specific deletion of the DD of CD95 in Tnfr1^{KO};Hoil-1^{E-KO} mice. Strikingly, this led to the complete prevention of dermatitis at D70 and to a significant prolonged survival in the resulting Cd95E-DD; Trail-r^{KO};Tnfr1^{KO};Hoil-1^{E-KO} mice as compared to Tnfr1^{KO};Hoil-1^{E-KO} mice (FIG. 4b-d).

We therefore conclude that CD95- and TRAIL-R-induced cell death can compensate for each other to drive inflammation in Tnfr1^{KO};Hoil-1^{E-KO} mice and that only when both systems are simultaneously inactivated, TNFR1-independent disease can be prevented.

We next studied the role of necroptosis in the pathology of Tnfr1^{KO}; Hoip^{E-KO} mice. Deletion of MLKL in Tnfr1^{KO};Hoip^{E-KO} mice significantly delayed the progression of dermatitis as Mlkl^{KO}; Tnfr1^{KO}; Hoip^{E-KO} mice had milder lesions at D70 as compared to Tnfr1^{KO}; Hoip^{E-KO} mice (FIG. 4b,c). However, these mice died at around D90 due to severe dermatitis (FIG. 4d). Hence, whilst necroptosis contributes to the TNFR1-independent disease, it is not solely responsible for it as the persisting apoptosis is sufficient to drive the disease.

### Example 8—Conclusions from Examples 2 to 7

Collectively, our study reveals a vital and previously unknown physiological role of HOIP and HOIL-1 in preventing lethal dermatitis. This skin inflammation is caused by the TNFR1-dependent, but importantly also by the TNFR1-independent death of keratinocytes (Extended Data FIG. 9c).

Furthermore, we identified that this TNFR1-independent cell death is driven by the orchestrated action of TRAIL and CD95L signalling systems.

These findings have several implications for the treatment of auto-inflammation and auto-immunity that go beyond the current treatment paradigm which is the inhibition of TNF.

Firstly, we identify prevention of cell death, regardless of the trigger, as a possibly effective strategy for the treatment of auto-immunity.

Secondly, our study provides evidence that combination treatments comprising blockers of TNF, TRAIL and CD95L may be of benefit to auto-immune patients who do not benefit from TNF inhibition alone and whose disease is currently categorized as refractory to TNF inhibition. Optionally in conjunction with RIPK1 kinase inhibition, or other inhibitors described herein, the methods of the invention may be expected to extend to diseases beyond auto-immune diseases which are currently amenable to treatment with TNF inhibitors.

### Methods for Examples 2-8

Mice. Hoip^{fl/fl} mice have been previously described²⁶. Hoil-1^{fl/fl} mice were generated by a gene targeting strategy in which the targeting cassette was composed of a hygromycin resistance cassette flanked by Frt sites and exons 1 and 2 of the Hoil-1 gene flanked by loxP sites. The hygromycin cassette was removed by crossing these mice with mice expressing the FlpE recombinase³⁴. To generate Hoip^{E-KO} and Hoil-1^{E-KO} mice Hoip^{fl/fl} and Hoil-1^{fl/fl} mice were crossed with mice expressing the Cre recombinase under the control of the human Keratin 14 promoter (obtained from Geert van Loo)¹², strain AZO-Nn4Cre (K14). Mlkl^{KO} mice were generated using TALEN technology. In brief, TALENs targeting exon 1 of the Mlkl gene were cloned via Golden-gate assembly. The RVD sequence of TAL1 against TACCGTTTCAGATGTCA was NI HD HD NN NG NG NG HD NI NN NI NG NN NG HD NI and TAL2 against TCGATCTTCCTGCTGCC was HD NN NI NG HD NG NG HD HD NG NN HD NG NN HD HD. Capped RNA was produced in vitro using mMESSAGE mMACHINE® T7 Transcription Kit (Ambion) and poly A tail was added using Poly(A) Tailing Kit (Ambion). Purified transcripts were mixed and adjusted to 25 ng/μL. Fertilised eggs were injected into both the cytoplasm and the pro-nucleus. Embryos were transferred into pseudo-pregnant females. Pups were genotyped by sequencing using genomic DNA obtained from ear punches. One female carrying a 19 bp homozygous deletion causing a premature stop codon was selected for further breeding. The K14CreER^{Tam} mice have been previously described³⁵. Tnfr1^{KO}, Tlr3^{KO} and Cd95-DD^{fl} mice (C57BL/6-Fastm1Cgn/J) were purchased from Jackson Laboratories. Ripk3^{KO 36}, Caspase-8^{KO 37} Trail-r^{KO} (Grosse-Wilde, A., Voloshanenko, O., Bailey, S. L., Longton, G. M., Schaefer, U., Csernok, A. I., Schutz, G., Greiner, E. F., Kemp, C. J., and Walczak, H. (2008). TRAIL-R deficiency in mice enhances lymph node metastasis without affecting primary tumor development. The Journal of clinical investigation 118, 100-110). and Ripk1^{D138N} mice have been previously described³⁸. To induce deletion of HOIP in the skin of adult mice Hoip^{fl/fl}K14CreER^{Tam} mice were treated as previously described³⁹. Briefly, a small shaved area of the dorsal neck was treated with 50 μL of 4-Hydroxytamoxifen (4-OHT) 20 mg/mL dissolved in ethanol every other day for a total of 1, 2, 3 or 4 treatments, as indicated. As vehicle treatment, a small dorsal area close to the tail was shaved and treated with ethanol. Hoip^{fl/wt}K14CreER^{Tam} mice were used as tamoxifen control. Mice were analysed 2 days after the last treatment or as indicated in the figure legends. Timed matings were performed as previously described²⁶. All mice were typed by PCR analysis. Colonies were fed ad libitum. All animal experiments were conducted under an appropriate UK project license in accordance with the regulations of UK home office for animal welfare according to ASPA (animal (scientific procedure) Act 1986).

Immunostaining and quantification. Four μm-thick formalin-fixed paraffin-embedded skin sections were stained following standard protocols. Briefly, sections were boiled in 10 mM sodium citrate buffer (pH 6.0) in a microwave. Slides were blocked in buffer containing Tween 20 0.5% and BSA 0.2%. For CD45 staining, slides were boiled in Retrievagen A (BD) and blocked with buffer without Tween. Next, slides were incubated with primary antibody overnight at 4° C. The following antibodies were used: anti-K14, anti-K10, anti-loricrin and anti-K6 (Covance), anti-Ki-67 (Abcam), anti-CD45 (BDbiosciences), anti-cleaved Caspase-3 (Cell Signaling), anti-HOIP (custom-made, Thermo Fisher Scientific), anti-HOIL-1¹¹. Slides were incubated with the following secondary antibodies: Alexa Fluor 488 Goat anti-Rabbit IgG, 594 Goat anti-Rabbit IgG (Invitrogen) or goat anti-rat HRP (Cambridge bioscience) at room temperature (RT) for 1 h. Where an HRP-conjugated antibody was used, the TSA™ Plus Cyanine 3 System (Perkin Elmer) was applied according to the manufacturer's instructions. Sections were counterstained with DAPI (Roche). Alternatively, conventional immunohistochemistry was performed on BOND-III (Leica Microsystems) and BenchMark Ultra (Ventana-Roche Medical System) according to a protocol previously described⁴⁰. For TUNEL staining, which was performed in combination with cleaved Caspase-3 staining, the ApopTag Red In Situ Apoptosis Detection kit (Merck Millipore) was used according to the manufacturer's instructions. Sections were analysed by fluorescent microscopy. At least ten different images (40×) per slide were acquired. Quantification was performed by an experimenter who was blinded to the genotype of the samples by using ImageJ Software on monochrome images as the percentage of cells positive for the specific staining in relation to the total number of cells (DAPI-positive) within the epidermis.

Epidermal thickness quantification. The epidermal thickness was measured in 5 different positions per microscopic field for at least 10 different fields per mouse. Quantification was performed by an experimenter who was blinded to the genotype of the samples by using ImageJ Software.

Scoring system. Mice were assessed macroscopically based on two main clinical criteria. Each region of the body, comprising head, neck, back and flank, affected by lesions, was given a score of 1 and the sum of these provided information of how expanded the lesions were. The other criteria was the character of the lesion: punctuated small crusts, coalescent crusts and ulceration, with a score 1 to 3, respectively. The sum of both criteria represented the total severity score of the lesions. Scoring was performed by two independent researchers.

Isolation, culture and viability of primary murine keratinocytes. PMKs were obtained from Hoip^{E-KO} newborn pups, Tnfr1^{KO};Hoip^{E-KO} and Tnfr1^{KO};Hoil-1^{E-KO} adult tails according to established protocols⁴¹. Briefly, skin was incubated with 0.25% Trypsin in HBSS without calcium and magnesium (Stratech Scientific Ltd) overnight at 4° C. The following day, dermis and epidermis were separated. Cell suspension was cultured in EMEM (Lonza) without calcium with 8% chelate FCS and penicillin-streptomycin (Sigma). PMKs were seeded in plates pre-coated with collagen I (Life technologies) for subsequent experiments. PMKs were cultured in medium supplemented with 20 μM Z-VAD-fmk (Abcam), 10 μM Necrostatin-1s (Cambridge Bioscience), 1 μM RIPK3 inhibitor (GSK2399872B) or 50 μg/mL Etanercept (Enbrel®) (Pfizer and Pentaglobin from Biotest) for four days, with supplemented medium replaced every day. On the last day, cell viability was measured using the CellTiter-Glo Luminescent Cell Viability Assay kit (Promega) following the manufacturers instructions. Alternatively, PMKs were treated for 24 hours with the following ligands as indicated: 50 ng/ml mouse iz-TRAIL, 50 ng/ml CD95L-Fc or 100 μg/ml Poly(I:C) (Invitrogen).

Western blotting and Immunoprecipitation. Western blotting was performed as previously described¹¹. Briefly, PMKs were lysed in IP-lysis buffer (30 mM Tris-HCl [pH 7.4], 120 mM NaCl, 2 mM EDTA, 2 mM KCl, 1% Triton X-100, EDTA-free proteinase inhibitor cocktail (Roche) and 1× phosphatase-inhibitor cocktail 2 (Sigma) at 4° C. for 20 min. Lysates were denatured with reducing sample buffer and DTT at 95° C. for 10 min. Proteins were separated by SDS-PAGE (NuPAGE) and analysed by Western blotting with antibodies against HOIP (custom-made, Thermo Fisher Scientific), HOIL-1¹¹, Sharpin (ProteinTech), actin (Sigma), tubulin (Sigma), FADD (Santa Cruz), RIPK1 (BD), cleaved Caspase-8 (Cell signalling), MLKL (Millipore), TNFR1 (Abcam), phosphorylated IκBα (Cell Signaling), IκBα (Cell Signaling) and linear ubiquitin (Millipore). Isolation of native TNFR1-SC and FADD immunoprecipitation (IP) were performed as previously described²⁶. Briefly, PMKs were cultured in the presence of 20 μM Z-VAD-fmk (Abcam) and, in the case of TNFR1-SC analysis, stimulated with 0.5 μg/mL 3×Flag-2×Strep-TNF for the indicated times or left untreated. Cellular lysates were subjected to anti-Flag IP using M2 beads (SIGMA; Schnelldorf, Germany) for 16 h. For FADD IP, lysates were incubated with anti-FADD antibody (Santa Cruz) and protein G Sepharose Beads (GE healthcare) at 4° C. for 4 h.

Flow cytometry. Cell suspensions obtained from skin samples were fluorescently labelled with Fixable Viability Dye eFluor® 780 (eBioscience). Samples were then stained with antibodies against the following cell surface markers: CD45-APC, CD45-AF700, CD3-PerCP/Cy5.5, CD4-FITC, CD8-PE/Cy7, GR1-FITC, GR1-PE/Cy7, F4/80-PE, F4/80-BV786, CD11b-Percp/Cy5.5 (Biolegend), CD19-BV650 and CD19-PE (Invitrogen). Samples were acquired with a LSRFORTESSA X-20 (BD) or Accuri (BD) with subsequent data analysis using FlowJo software.

Statistics. Data were analysed with GraphPad Prism 6 software (GraphPad Software) or Microsoft Excel. Data shown in graphs represent the mean values±s.e.m, as indicated in the figure legends. Preliminary data sets were used to determine the group size necessary for adequate statistical power. Statistical analyses were performed by unpaired two tailed Student's t test. Statistical significance in survival curves was determined using a log-rank test. A P value of >0.05 was considered not significant (NS), whereas 0.05 was indicated with one asterisk (*), P≤0.01 (**) and P≤0.001 (***). In all cases comparisons were made between the indicated KO mice and the respective littermate controls.

### REFERENCES FOR EXAMPLES 2-8

7 Gerlach, B. et al. Linear ubiquitination prevents inflammation and regulates immune signalling. Nature 471, 591-596, doi:10.1038/nature09816 (2011).
8 Rickard, J. A. et al. TNFR1-dependent cell death drives inflammation in Sharpin-deficient mice. eLife 3, doi:10.7554/eLife.03464 (2014).
10 Kumari, S. et al. Sharpin prevents skin inflammation by inhibiting TNFR1-induced keratinocyte apoptosis. eLife 3, doi:10.7554/eLife.03422 (2014).
11 Haas, T. L. et al. Recruitment of the linear ubiquitin chain assembly complex stabilizes the TNF-R1 signaling complex and is required for TNF-mediated gene induction. Molecular cell 36, 831-844, doi:10.1016/j.molcel.2009.10.013 (2009).
13 Tokunaga, F. et al. Involvement of linear polyubiquitylation of NEMO in NF-kappaB activation. Nat Cell Biol, doi:10.1038/ncb1821 (2009).
19 Tokunaga, F. Linear ubiquitination-mediated NF-kappaB regulation and its related disorders. Journal of biochemistry 154, 313-323, doi:10.1093/jb/mvt079 (2013).
20 Shimizu, Y., Taraborrelli, L. & Walczak, H. Linear ubiquitination in immunity. Immunological reviews 266, 190-207, doi:10.1111/imr.12309 (2015).
21 Zinngrebe, J. et al.—LUBAC deficiency perturbs TLR3 signaling to cause immunodeficiency and autoinflammation. The Journal of experimental medicine, doi:10.1084/jem.20160041 (2016).
22 HogenEsch, H. et al. A spontaneous mutation characterized by chronic proliferative dermatitis in C57BL mice. The American journal of pathology 143, 972-982 (1993).
23 HogenEsch, H., Janke, S., Boggess, D. & Sundberg, J. P. Absence of Peyer's patches and abnormal lymphoid architecture in chronic proliferative dermatitis (cpdm/cpdm) mice. J Immunol 162, 3890-3896 (1999).
24 Seymour, R. E. et al. Spontaneous mutations in the mouse Sharpin gene result in multiorgan inflammation, immune system dysregulation and dermatitis. Genes Immun 8, 416-421, doi:6364403 [pii]10.1038/sj.gene.6364403 (2007).
25 Berger, S. B. et al. Cutting Edge: RIP1 kinase activity is dispensable for normal development but is a key regulator of inflammation in SHARPIN-deficient mice. J Immunol 192, 5476-5480, doi:10.4049/jimmunol.1400499 (2014).
26 Peltzer, N. et al. HOIP deficiency causes embryonic lethality by aberrant TNFR1-mediated endothelial cell death. Cell Rep 9, 153-165, doi:10.1016/j.celrep.2014.08.066 (2014).
27 Tokunaga, F. et al. Involvement of linear polyubiquitylation of NEMO in NF-kappaB activation. Nature cell biology 11, 123-132, doi:10.1038/ncb1821 (2009).
28 Peltzer, N., Darding, M. & Walczak, H. Holding RIPK1 on the Ubiquitin Leash in TNFR1 Signaling. Trends in cell biology, doi:10.1016/j.tcb.2016.01.006 (2016).
29 Kaiser, W. J. et al. RIP3 mediates the embryonic lethality of caspase-8-deficient mice. Nature 471, 368-372, doi:10.1038/nature09857 (2011).
30 Oberst, A. et al. Catalytic activity of the caspase-8-FLIP(L) complex inhibits RIPK3-dependent necrosis. Nature 471, 363-367, doi:10.1038/nature09852 (2011).
32 Lafont, E. et al. The linear ubiquitin chain assembly complex regulates TRAIL-induced gene activation and cell death. The EMBO journal, doi:10.15252/embj.201695699 (2017).
34 Rodriguez, C. I. et al. High-efficiency deleter mice show that FLPe is an alternative to Cre-loxP. Nature genetics 25, 139-140, doi:10.1038/75973 (2000).
35 Vasioukhin, V., Degenstein, L., Wise, B. & Fuchs, E. The magical touch: genome targeting in epidermal stem cells induced by tamoxifen application to mouse skin. Proceedings of the National Academy of Sciences of the United States of America 96, 8551-8556 (1999).
Newton, K., Sun, X. & Dixit, V. M. Kinase RIP3 is dispensable for normal NF-kappa Bs, signaling by the B-cell and T-cell receptors, tumor necrosis factor receptor 1, and Toll-like receptors 2 and 4. Molecular and cellular biology 24, 1464-1469 (2004).
37 Salmena, L. et al. Essential role for caspase 8 in T-cell homeostasis and T-cell-mediated immunity. Genes & development 17, 883-895, doi:10.1101/gad.1063703 (2003).
38 Polykratis, A. et al. Cutting edge: RIPK1 Kinase inactive mice are viable and protected from TNF-induced necroptosis in vivo. J Immunol 193, 1539-1543, doi:10.4049/jimmunol.1400590 (2014).
39 Weinlich, R. et al. Protective Roles for Caspase-8 and cFLIP in Adult Homeostasis. Cell reports, doi:http://dx.doi.org/10.1016/j.celrep.2013.08.045 (2013).
40 Marafioti, T. et al. Novel markers of normal and neoplastic human plasmacytoid dendritic cells. Blood 111, 3778-3792, doi:10.1182/blood-2007-10-117531 (2008).
41 Lichti, U., Anders, J. & Yuspa, S. H. Isolation and short-term culture of primary keratinocytes, hair follicle populations and dermal cells from newborn mice and keratinocytes from adult mice for in vitro analysis and for grafting to immunodeficient mice. Nat Protoc 3, 799-810, doi:10.1038/nprot.2008.50 (2008).

### REFERENCES FOR TABLES 2 AND 3

Arnason (1999). TNF neutralization in MS: results of a randomized, placebo-controlled multicenter study. The Lenercept Multiple Sclerosis Study Group and The University of British Columbia MS/MRI Analysis Group. Neurology 53, 457-465.
Cessak, G., Kuzawinska, O., Burda, A., Lis, K., Wojnar, M., Mirowska-Guzel, D., and Balkowiec-Iskra, E. (2014). TNF inhibitors—Mechanisms of action, approved and off-label indications. Pharmacol Rep 66, 836-844.
Boisson, B., Laplantine, E., Prando, C., Giliani, S., Israelsson, E., Xu, Z., Abhyankar, A., Israel, L., Trevejo-Nunez, G., Bogunovic, D., et al. (2012). Immunodeficiency, autoinflammation and amylopectinosis in humans with inherited HOIL-1 and LUBAC deficiency. Nat Immunol 13, 1178-1186.
Chaudhari, U., Romano, P., Mulcahy, L. D., Dooley, L. T., Baker, D. G., and Gottlieb, A. B. (2001). Efficacy and safety of infliximab monotherapy for plaque-type psoriasis: a randomised trial. Lancet 357, 1842-1847.
Cheng, K., Wang, X., and Yin, H. (2011). Small-molecule inhibitors of the TLR3/dsRNA complex. J Am Chem Soc 133, 3764-3767.
Cho, J. H., and Feldman, M. (2015). Heterogeneity of autoimmune diseases: pathophysiologic insights from genetics and implications for new therapies. Nature medicine 21, 730-738.
Croft, M., and Siegel, R. M. (2017). Beyond TNF: TNF superfamily cytokines as targets for the treatment of rheumatic diseases. Nature reviews. Rheumatology 13, 217-233.
Damgaard, R. B., Walker, J. A., Marco-Casanova, P., Morgan, N. V., Titheradge, H. L., Elliott, P. R., McHale, D., Maher, E. R., McKenzie, A. N. J., and Komander, D. (2016). The Deubiquitinase OTULIN Is an Essential Negative Regulator of Inflammation and Autoimmunity. Cell 166, 1215-1230 e1220.
Fausel, R., and Afzali, A. (2015). Biologics in the management of ulcerative colitis —comparative safety and efficacy of TNF-alpha antagonists. Ther Clin Risk Manag 11, 63-73.
Fauster, A., Rebsamen, M., Huber, K. V., Bigenzahn, J. W., Stukalov, A., Lardeau, C. H., Scorzoni, S., Bruckner, M., Gridling, M., Parapatics, K., et al. (2015). A cellular screen identifies ponatinib and pazopanib as inhibitors of necroptosis. Cell death & disease 6, e1767.
Fernandez-Nebro, A., Olive, A., Castro, M. C., Varela, A. H., Riera, E., Irigoyen, M. V., Garcia de Yebenes, M. J., and Garcia-Vicuna, R. (2010). Long-term TNF-alpha blockade in patients with amyloid A amyloidosis complicating rheumatic diseases. Am J Med 123, 454-461.
Gao W, Xiong Y, Li Q and Yang H (2017) Inhibition of Toll-Like Receptor Signaling as a Promising Therapy for Inflammatory Diseases: A Journey from Molecular to Nano Therapeutics. Front. Physiol. 8:508. doi: 10.3389/fphys.2017.00508
Hanauer, S. B., Feagan, B. G., Lichtenstein, G. R., Mayer, L. F., Schreiber, S., Colombel, J. F., Rachmilewitz, D., Wolf, D. C., Olson, A., Bao, W., et al. (2002). Maintenance infliximab for Crohn's disease: the ACCENT I randomised trial. Lancet 359, 1541-1549.
Harris, P. A., Berger, S. B., Jeong, J. U., Nagilla, R., Bandyopadhyay, D., Campobasso, N., Capriotti, C. A., Cox, J. A., Dare, L., Dong, X., et al. (2017). Discovery of a First-in-Class Receptor Interacting Protein 1 (RIP1) Kinase Specific Clinical Candidate (GSK2982772) for the Treatment of Inflammatory Diseases. J Med Chem 60, 1247-1261.
Hoglen, N. C., Chen, L. S., Fisher, C. D., Hirakawa, B. P., Groessl, T., and Contreras, P. C. (2004). Characterization of IDN-6556 (3-[2-(2-tert-butyl-phenylaminooxalyl)-amino]-propionylamino]-4-oxo-5-(2,3,5,6-tetrafluoro-phenoxy)-pentanoic acid): a liver-targeted caspase inhibitor. J Pharmacol Exp Ther 309, 634-640.
Jia, Z., Xu, C., Shen, J., Xia, T., Yang, J., and He, Y. (2015). The natural compound celastrol inhibits necroptosis and alleviates ulcerative colitis in mice. Int Immunopharmacol 29, 552-559.
Kearsley-Fleet, L., McErlane, F., Foster, H. E., Lunt, M., Watson, K. D., Symmons, D. P., and Hyrich, K. L. (2016). Effectiveness and safety of TNF inhibitors in adults with juvenile idiopathic arthritis. RMD Open 2, e000273.
Lee, R. A., and Eisen, D. B. (2015). Treatment of hidradenitis suppurativa with biologic medications. J Am Acad Dermatol 73, S82-88.
Li, D., Li, C., Li, L., Chen, S., Wang, L., Li, Q., Wang, X., Lei, X., and Shen, Z. (2016). Natural Product Kongensin A is a Non-Canonical HSP90 Inhibitor that Blocks RIP3-dependent Necroptosis. Cell Chem Biol 23, 257-266.
Liu, W., Wu, Y. H., Zhang, L., Liu, X. Y., Bin, X., Bin, L., Wang, Y., and Ji, Y. (2016). Efficacy and safety of TNF-alpha inhibitors for active ankylosing spondylitis patients: Multiple treatment comparisons in a network meta-analysis. Scientific reports 6, 32768.
Manns, M., Bichler, Z., Leske, O., and Heumann, R. (2010). Neuronal Ras activation inhibits adult hippocampal progenitor cell division and impairs spatial short-term memory. Genes Brain Behav 9, 525-536.
Mease, P. J. (2002). Tumour necrosis factor (TNF) in psoriatic arthritis: pathophysiology and treatment with TNF inhibitors. Ann Rheum Dis 61, 298-304.
Meijer, J. M., Pijpe, J., Bootsma, H., Vissink, A., and Kallenberg, C. G. (2007). The future of biologic agents in the treatment of Sjogren's syndrome. Clin Rev Allergy Immunol 32, 292-297.
Ratziu, V., Sheikh, M. Y., Sanyal, A. J., Lim, J. K., Conjeevaram, H., Chalasani, N., Abdelmalek, M., Bakken, A., Renou, C., Palmer, M., et al. (2012). A phase 2, randomized, double-blind, placebo-controlled study of GS-9450 in subjects with nonalcoholic steatohepatitis. Hepatology 55, 419-428.
Roda, G., Jharap, B., Neeraj, N., and Colombel, J. F. (2016). Loss of Response to Anti-TNFs: Definition, Epidemiology, and Management. Clin Transl Gastroenterol 7, e135.
St Clair, E. W., Baer, A. N., Noaiseh, G., Parke, A., Coca, A., Utset, T., Genovese, M. C., Wallace, D. J., McNamara, J., Boyle, K., et al. (2015). The Clinical Efficacy and Safety of Baminercept, a Lymphotoxin-Beta Receptor Fusion Protein, in Primary Sjogren's Syndrome: Results from a Randomized, Double-Blind, Placebo-Controlled Phase II Trial. Arthritis & Rheumatology 67.
Stohl, W. (2013). Future prospects in biologic therapy for systemic lupus erythematosus. Nature reviews. Rheumatology 9, 705-720.
Tweedie, D., Sambamurti, K., and Greig, N. H. (2007). TNF-alpha inhibition as a treatment strategy for neurodegenerative disorders: new drug candidates and targets. Curr Alzheimer Res 4, 378-385.
Wick, W., Fricke, H., Junge, K., Kobyakov, G., Martens, T., Heese, O., Wiestler, B., Schliesser, M. G., von Deimling, A., Pichler, J., et al. (2014). A phase II, randomized, study of weekly APG101+reirradiation versus reirradiation in progressive glioblastoma. Clinical cancer research: an official journal of the American Association for Cancer Research 20, 6304-6313.
Yang, H., Kozicky, L., Saferali, A., Fung, S. Y., Afacan, N., Cai, B., Falsafi, R., Gill, E., Liu, M., Kollmann, T. R., et al. (2016). Endosomal pH modulation by peptide-gold nanoparticle hybrids enables potent anti-inflammatory activity in phagocytic immune cells. Biomaterials 111, 90-102.

### PATENT REFERENCES

U.S. Pat. No. 5,919,452 A
EP 0914157 B1
WO 2013087912 A1
WO 2007022813 A2
EP 1447093 A1
WO 2004071528 A1
EP 1020521 A1
WO 2010006772 A3
WO2015001345
U.S. Pat. No. 8,153,583 B2

1. A method for treating inflammatory disease in a subject, the method comprising administering to the subject a combination treatment of at least 3 agents, the combination comprising:
(1) a first agent that neutralises the receptor TNFR1 or a ligand thereof;
and
(2) a second agent that neutralises either of:
(2a) TRAIL-R, or a ligand thereof;
or
(2b) CD95, or a ligand thereof;
and:
(3) a third agent that neutralises any of:
(3a) TLR3, or TLR4, or a ligand of either; or
(3b) a further, different, receptor which is a TNF Receptor superfamily member shown in Table 1, or a ligand thereof;
(3c) Caspase;
(3d) RIPK1.

2. A method of enhancing the therapeutic effectiveness of:
(1) a first agent that neutralises the receptor TNFR1 or a ligand thereof;
for treating an inflammatory disease in a subject, the method comprising administering to the subject:
(2) a second agent that neutralises either of:
(2a) TRAIL R, or a ligand thereof;
or
(2b) CD95, or a ligand thereof;
and:
(3) a third agent that neutralises any of:
(3a) TLR3, or TLR4, or a ligand of either; or
(3b) a further, different, receptor which is a TNF Receptor superfamily member shown in Table 1, or a ligand thereof;
(3c) Caspase;
(3d) RIPK1.

3. The method of claim 1 or claim 2, wherein the agent that neutralises a receptor or ligand thereof, either:
(i) prevents or inhibits the ligand from binding to the receptor;
(ii) disrupts the receptor/ligand complex resulting from such binding.

4. The method of any one of claims 1 to 3 wherein the first agent neutralises TNF and\or LT-α.

5. The method of any one of claims 1 to 4 wherein the second agent neutralises a TRAIL-R, or neutralises TRAIL.

6. The method of claim 5 wherein the second agent neutralises TRAIL-R1 and\or TRAIL R2.

7. The method of claim 5 or claim 6 wherein the third agent neutralises CD95, or neutralises CD95L.

8. The method of claim 6 or claim 7 wherein:
(1) the first agent neutralises TNF and\or LT-α;
(2a) the second agent neutralises a TRAIL-R or TRAIL;
(3a) the third agent neutralises CD95L.

9. The method of any one of claims 1 to 5 wherein the third agent neutralises TLR3, or neutralises a ligand of TLR3, or TLR4, or a ligand of either

10. The method of claim 9 wherein:
(1) the first agent neutralises TNF and\or LT-α;
(2a) the second agent neutralises TRAIL-R or TRAIL;
(3a) the third agent neutralises TLR3.

11. The method of any one of claims 1 to 4 wherein the second agent neutralises CD95, or neutralises CD95L.

12. The method of claim 11 wherein the third agent neutralises TLR3, or neutralises a ligand of TLR3.

13. The method of claim 12 wherein:
(1) the first agent neutralises TNF and\or LT-α;
(2a) the second agent neutralises CD95 or CD95L;
(3a) the third agent neutralises TLR3.

14. The method of any one of claims 1 to 5 or claim 11 wherein the third agent neutralises Caspase, and a fourth agent is used which neutralises RIPK3 and\or MLKL.

15. The method of claim 14, wherein the caspase is Caspase 8.

16. The method of any one of claims 1 to 5 or claim 11 wherein the third agent neutralises LT-β.

17. The method of any one of claims 1 to 5 or claim 11 wherein the third agent neutralises RIPK1.

18. The method of any one of claims 1 to 17, wherein the agent is a single or double-stranded nucleotide (DNA, RNA(siRNA, miRNA, shRNA), PNA, DNA-RNA-hybrid molecule) that interferes with expression of the receptor or ligand or is an antibody or fragment thereof that binds to and neutralises the receptor or ligand

19. The method of any one of claims 1 to 13 which utilises one or more of the inhibitors shown in Table 2.

20. The method of any one of claims 5 to 10 or 14 to 17 which utilises a second agent which decreases the biological activity of a TRAIL-R or TRAIL by:
(a) decreasing the expression of the receptor;
(b) increasing receptor desensitisation or receptor breakdown;
(c) reducing interaction between TRAIL and the receptor which is an endogenous receptor;
(d) reducing receptor mediated intracellular signalling;
(e) competes with endogenous receptor for TRAIL binding;
(f) binds to the receptor to block TRAIL binding; or
(g) binds to TRAIL preventing interaction with the receptor.

21. The method of claim 20 which utilises a second agent that binds to and neutralises TRAIL.

22. The method of claim 21, wherein the agent is an antibody or fragment thereof that binds to and neutralises TRAIL.

23. The method of claims 20 to 21 which utilises an agent which is a fusion protein comprising an extracellular domain of a TRAIL-R, preferably of TRAIL-R2, or a portion thereof, fused to a human antibody Fc domain, or a portion thereof, with or without the antibody hinge region, or a portion thereof.

24. The method of claim 20 which utilises a second agent that binds to two or more TRAIL-Rs, or wherein the method utilises a second agent and one or more further agents, each of which binds to one or more TRAIL-Rs.

25. The method of claim 23, wherein the second agent:
(1) is an antibody or fragment thereof that binds to both TRAIL-R1 and TRAIL-R2, neutralising their activity, or wherein
(2) the second agent is an antibody or fragment thereof that binds to TRAIL-R1 neutralising its activity, and is used with a further antibody or fragment thereof which binds TRAIL-R2 neutralising its activity.

26. The method of any one of claims 1 to 25 which further comprises administering to the subject one or more agents, or one or more further agents which neutralise a mediator of extrinsic apoptosis and\or necroptosis, which is optionally selected from one or more of: Caspase, RIPK3 and MLKL.

27. The method of any one of claims 1 to 26 which further comprises administering to the subject a further anti-inflammatory biologic or anti-inflammatory chemical agent.

28. The method of claim 27 wherein the further anti-inflammatory biologic or chemical agent is an oral or topical corticosteroid.

29. The method of any one of claims 1 to 28 wherein the inflammatory disease is selected from the list consisting of: an auto-immune disease optionally selected from multiple sclerosis (MS), amyotrophic lateral sclerosis (ALS); a neuro-inflammatory disease, which is optionally muscular dystrophy; a neuro-degenerative disease optionally selected from Parkinson's Disease, Alzheimer's Disease, and Huntington's Disease; an ischaemic disease optionally selected from ischaemic diseases of the heart, the kidney or the brain; sepsis; an inflammatory disease caused by any of HOIL-1, HOIP or OTULIN deficiencies.

30. The method of any one of claims 1 to 29 wherein the inflammatory disease is selected from Table 3.

31. The method of claim 30 wherein the inflammatory disease is selected from the list consisting of rheumatoid arthritis (RA); psoriasis; inflammatory bowel disease (IBD).

32. The method of any one of claims 1 to 28 wherein the inflammatory disease is a cancer, and the method further comprises administering to the subject one or more additional agents for treating said cancer or performing radiotherapy on said subject.

33. The method of claim 32, wherein the one or more additional agents for treating said cancer are selected from the lists consisting of chemotherapeutics; immune checkpoint inhibitors optionally selected from anti-PD-1/L1 and/or anti-CTLA-4 antibodies; cell-based therapies optionally selected from such as transgenic chimaeric antigen receptor (CAR)- or T cell receptor (TCR)-expressing T cells.

34. The method of any one of claims 1 to 33 wherein the subject is selected as one having an inflammatory disease, and further selected by screening for evidence of cell death in biological sample taken from said patient.

35. The method of any one of claims 1 to 34 wherein the subject is selected as one having an inflammatory disease, and in whom the disease has proved refractory to treatment with a TNF inhibitor.

36. The method of claim 35 comprising the steps of
(i) selecting an subject in whom the disease has proved refractory to treatment with a TNF inhibitor.
and
(ii) administering to the subject said combination treatment of at least 3 agents.

37. A first agent that neutralises the receptor TNFR1 or neutralises a ligand of TNFR1, for use in a combination method of any one of claims 1 to 36.

38. A second agent that neutralises either of:
(2a) TRAIL R, or a ligand thereof;
or
(2b) CD95, or a ligand thereof;
for use in a combination method of any one of claims 1 to 36.

39. A third agent that neutralises any of:
(3a) TLR3, or TLR4, or a ligand of either; or
(3b) a further, different, receptor which is TNF Receptor superfamily member shown in Table 1, or a ligand thereof;
(3c) Caspase;
(3d) RIPK1, for use in a combination method of any one of claims 1 to 36.

40. A combination treatment of at least 3 agents, the combination comprising:
(1) a first agent that neutralises the receptor TNFR1 or a ligand thereof;
and
(2) a second agent that neutralises either of:
(2a) TRAIL R, or a ligand thereof;
or
(2b) CD95, or a ligand thereof;
and:
(3) a third agent that neutralises any of:
(3a) TLR3, or TLR4, or a ligand of either; or
(3b) a further, different, receptor which is TNF Receptor superfamily member shown in Table 1, or a ligand thereof;
(3c) Caspase;
(3d) RIPK1, which combination treatment is for use in a method for treating inflammatory disease
in a subject,
the method comprising administering to the subject said combination treatment of at least 3 agents.

41. Use of:
(1) a first agent that neutralises the receptor TNFR1 or a ligand thereof;
and
(2) a second agent that neutralises either of:
(2a) TRAIL R, or a ligand thereof;
or
(2b) CD95, or a ligand thereof;
and:
(3) a third agent that neutralises any of:
(3a) TLR3, or TLR4, or a ligand of either; or
(3b) a further, different, receptor which is a TNF Receptor superfamily member shown in Table 1, or a ligand thereof;
(3c) Caspase;
(3d) RIPK1.
in the manufacture of a medicament for treatment of inflammatory disease
in a subject,

42. Use of
a second agent that neutralises either of:
(2a) TRAIL R, or a ligand thereof;
or
(2b) CD95, or a ligand thereof;
and:
in the manufacture of a medicament for treatment of inflammatory disease
in a subject, which treatment further comprises use of:
(1) a first agent that neutralises the receptor TNFR1 or a ligand thereof;
and
and:
(3) a third agent that neutralises any of:
(3a) TLR3, or TLR4, or a ligand of either; or
(3b) a further, different, receptor which is a TNF Receptor superfamily member shown in Table 1, or a ligand thereof;
(3c) Caspase;
(3d) RIPK1.

43. The combination treatment or use of any one of claims 39 to 42 for use in the method of any one of claims 1 to 36, or wherein the or each agent is an agent as defined in any of those claims.

44. A method, treatment or use of any one of claims 1 to 43, wherein the first agent, the second agent and the third agent are administered sequentially within 12 hours of each other.

45. A method, treatment or use of any one of claims 1 to 43, wherein the first agent, the second agent and the third agent are administered simultaneously, optionally within a single dosage unit.


SEQUENCE LISTING (5 sequences)

SEQ ID NO: 1 - protein, 7 aa, Artificial Sequence
  TRAIL-R2-Fx polypeptide mature protein
ITQQDLA

SEQ ID NO: 2 - protein, 435 aa, Artificial Sequence
  TRAIL-R2-Fc leader peptide
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 9 aa, Artificial Sequence
  Hydrophilic residues
GPSNDQERK

SEQ ID NO: 4 - DNA, 17 bp, Artificial Sequence
  TAL1 target sequence
taccgtttcagatgtca

SEQ ID NO: 5 - DNA, 17 bp, Artificial Sequence
  TAL2 target sequence
tcgatcttcctgctgcc